(19) United States
(12) Patent Application Publication
(10) Pub. No.: US 2026/0216231 A1
(43) Pub. Date: Jul. 30, 2026

(54) PERIODONTAL TREATMENT METHOD AND COMPOSITION

(71) Applicant: Forward Science Technologies, LLC, Houston, TX (US)

(72) Inventors: Brian PIKKULA, Sugar Land, TX (US); Robert WHITMAN, Houston, TX (US); Delaney PIERSON, Houston, TX (US); Jeremy POIRIER, Houston, TX (US); Naomi BIER, Houston, TX (US); Brian DORSEY, Houston, TX (US)

(73) Assignee: PerioStom LLC, Houston, TX (US)

(21) Appl. No.: 19/141,905

(22) PCT Filed: Dec. 22, 2023
(86) PCT No.: PCT/US2023/085741

§ 371 Date: Jun. 20, 2025

Related U.S. Application Data
(60) Provisional application No. 63/476,855, filed on Dec. 22, 2022.

(51) Int. Cl. A61K 31/722; A61K 9/00; A61K 9/14

(52) U.S. Cl. CPC A61K 31/722; A61K 9/0063; A61K 9/146

(57) ABSTRACT

The invention provides a composition, e.g., in the form of a powder, comprising chitosan particles. The invention also provides method for treating an oral condition in a patient by administering to the patient a composition of the invention to treat the oral condition. The invention further provides devices suitable for administering the composition.

### CROSS-REFERENCE TO RELATED APPLICATION

This patent application claims the benefit of U.S. Provisional Patent Application No. 63/476,855, filed Dec. 22, 2022, which is incorporated herein by reference in its entirety.

### BACKGROUND OF THE INVENTION

In dental practice, medical professionals are often faced with the need to treat wounds in the oral cavity. Trauma-related oral wounds are encountered frequently in clinical dental practice. Wounds in the oral cavity can impair a patient's normal oral function, and may cause pain in a patient's eating, chewing, and speaking. An injury to the oral mucosa can result from, e.g., physical, chemical, or thermal trauma, or from disease, e.g., infection. Such injuries can result, for example, from accidental tooth bite, hard foods, sharp edges of the teeth, hot food, excessive tooth brushing, and exposure to chemicals that are damaging to oral tissues. Some injuries also can be caused by iatrogenic damage during dental treatment, surgery, or other procedures performed within the oral cavity. Most traumatic oral wounds such as, e.g., sores, lesions, or ulcers in the oral cavity, may heal on their own without complication. However, in some cases persistent traumatic factors such as, for example, sharp tooth morphology, sharp edges on dental restorations, and puncturing appliance contours, e.g., from inadequate prosthetic surfaces, can cause continuous trauma which can lead to formation of chronic ulcers.

Common strategies for treating oral wounds include pain relief, infection control, and acceleration/promotion of wound repair. The most common for treating oral wounds is pain relief. Indeed, some sources have reported that over 30% of all opioids are prescribed by dentists and oral surgeons. Topical dressings also may be used for oral wound therapy, and are among the preferred protocols as they have considerably lower risk of adverse effects than other protocols such as systemic drugs, surgical excision, and laser treatment. By providing a safer alternative for pain management, therapeutic wound dressings may indirectly help in combatting the spread of opioid addiction.

Oral wound dressings are known in the art and sold commercially for the topical treatment of wounds in the oral cavity. For example, Arestin® is a subgingival sustained-release product containing the antibiotic minocycline hydrochloride incorporated into a bioresorbable polymer, polyglycolide-co-dl-lactide or PGLA, for professional subgingival administration into periodontal pockets. Arestin® is used as an adjunct to scaling and root planning procedures for reduction of pocket depth in patients with adult periodontitis. Another example, Atridox®, is a subgingival controlled-release product comprises of a two syringe mixing system. Atridox® contains doxycycline hyclate incorporated into a bioresorbable polymer, poly-dl-lactide or PLA, for professional subgingival administration into periodontal pockets. However, due to Atridox® being in gel form, there may be difficulties associated with the Atridox® gel such as, e.g., the ability for the gel to stay in the periodontal pocket.

Thus, there remains a need for new oral compositions that are safe and effective in treating wounds in the oral cavity, and which have improved properties, e.g., stability, shelf life, microbial resistance, and/or retention in the periodontal pocket. The present invention provides such compositions and methods of using them for the treatment of wounds, e.g., sores, lesions, and ulcers, in the oral cavity, as well as are suitable for use as an adjunct to scaling and root planning procedures for reduction of pocket depth in patients with adult periodontitis. These and other advantages of the invention, as well as additional inventive features, will be apparent from the description of the invention provided herein.

### BRIEF SUMMARY OF THE INVENTION

The present invention provides a composition, which may be in the form of a powder, e.g., a dry powder, comprising chitosan particles having a particle size distribution with a mode of from about 0.1 μm to about 100 μm.

The present invention also provides a method for treating an oral (e.g., dental) condition in a patient, the method comprising administering to the patient (e.g., subgingivally, topically, or sub-dermally, e.g., by introducing a sterile form of the composition inside of an oral surgical wound prior to suturing such that the composition resides inside the wound after suturing) a therapeutically effective amount of the composition of the invention, to treat the condition.

In some embodiments, the chitosan in the composition of the invention has a degree of deacetylation of from about 50% to about 100%, from about 80% to about 100%, from about 80% to about 99%, or from about 85% to about 99% relative to chitin.

In some embodiments, the method includes administering the composition of the invention by way of a unit dose cartridge comprising a stainless needle attached to a plastic tip (e.g., a ridged tip) or a metal tip, wherein the tip is inserted into the periodontal pocket or wound and the composition is expelled through the tip to administer the composition. For example, the composition of the invention may be administered with an applicator comprising any combination of at least one portion of the features described in FIGS. 1-8.

The present invention further provides an applicator comprising any combination of at least one portion of the features described in FIGS. 1-8.

### BRIEF DESCRIPTION OF THE DRAWINGS

The invention is best understood from the following detailed description when read in conjunction with the accompanying drawings. According to common practice, the dimensions and other various features of the drawings are not necessarily to-scale, and may be arbitrarily expanded or reduced for clarity.

FIG. 1A is a side elevational view of a syringe for administration of a medicament to a surface.

FIG. 1B is a plan view of the syringe of FIG. 1A

FIG. 1C is a cross-sectional view of the syringe of FIGS. 1A and 1B taken along line A-A in FIG. 1B.

FIG. 1D is an isometric view of the syringe of FIGS. 1A-1C.

FIG. 1E is an exploded isometric view of the syringe of FIGS. 1A-1D.

FIG. 2A is a plan view of an exemplary embodiment of syringe for administration of a medicament to a surface.

FIG. 2B is a cross-sectional view of the syringe of FIG. 2A taken along line B-B in FIG. 2A.

FIG. 2C is a cross-sectional view of the syringe of FIG. 2B with a drive stick and an actuator of the syringe shown in a delivered position for actuator travel of at least one embodiment of the syringe of FIGS. 1A and 1B.

FIG. 2D is a plan view of a tip assembly of the syringe of FIGS. 2A-2C.

FIG. 2E is a cross-sectional view of the tip assembly taken along line C-C in FIG. 2D.

FIG. 2F is a cross-sectional view of the tip assembly of FIG. 2E with an actuator of the tip shown in a delivered position for actuator travel of at least one embodiment of the syringe of FIGS. 1A and 1B.

FIG. 3A is an end view of an exemplary barrel of the syringes of FIGS. 1A-2C.

FIG. 3B is a side elevational view of the barrel of FIG. 3A

FIG. 3C is a cross-sectional view of the barrel of FIGS. 3A and 3B taken along line A-A in FIG. 3B.

FIG. 3D is a side elevational view of the barrel of FIGS. 3A-3C.

FIG. 3E is an isometric view of the barrel of FIGS. 3A-3D.

FIG. 4A is an end view of an exemplary actuator of the syringes of FIGS. 1A-2C.

FIG. 4B is a side elevational view of the actuator of FIG. 4A.

FIG. 4C is a cross-sectional view of the actuator of FIGS. 4A and 4B taken along line D-D in FIG. 4B.

FIG. 4D is an end view of the actuator of FIGS. 4A-4C taken from the opposite end from FIG. 4A.

FIG. 4E is a side elevational view of the actuator of FIGS. 4A-4D assembled with an exemplary needle.

FIG. 4F is an isometric view of the actuator of FIGS. 4A-4E.

FIG. 5A is an end view of an exemplary drive stick/plunger of the syringes of FIGS. 1A-2C.

FIG. 5B is a side elevational view of the drive stick/plunger of FIG. 5A.

FIG. 5C is a plan view of the drive stick/plunger of FIGS. 5A-5B.

FIG. 5D is an end view of the drive stick/plunger of FIGS. 5A-5C taken from the opposite end from FIG. 5A.

FIG. 5E is an isometric view of the drive stick/plunger of FIGS. 5A-5D.

FIG. 6A is an end view of a tip of a syringe of FIGS. 1A-2C, and the tip and needle/conduit of FIGS. 2D-2F.

FIG. 6B is a side elevational view of the tip of FIG. 6A.

FIG. 6C is a plan view of a tip of FIG. 6A-6B.

FIG. 6D is a cross-sectional view of the tip of FIGS. 6A-6C taken along line A-A in FIG. 6C.

FIG. 6E is an end view of the tip of FIGS. 6A-6D, taken from the end opposite that of FIG. 6A.

FIG. 6F is an isometric view of the tip of FIGS. 6A-6E.

FIG. 7A is an end view of an exemplary actuator probe of the syringe and the tip assembly of FIGS. 1A-2F.

FIG. 7B is a side elevational view of the actuator probe of FIG. 7A.

FIG. 7C is an isometric view of the actuator probe of FIGS. 7A and 7B.

FIG. 8A is an end view of an exemplary needle/conduit of the syringe and tip assembly of FIGS. 1A-2F.

FIG. 8B is a side elevational view of the needle/conduit of FIG. 8A.

FIG. 8C is an isometric view of the needle/conduit of FIGS. 8A and 8B.

FIG. 9 depicts a graph showing the particle size distribution of an exemplary powder comprising chitosan particles.

FIG. 10A is a side elevational view of an alternative embodiment of a syringe for administration of a medicament to a surface.

FIG. 10B is an end view of the syringe of FIG. 10A

FIG. 10C is a cross-sectional view of the syringe of FIGS. 10A and 10B taken along line A-A in FIG. 10B.

### DETAILED DESCRIPTION OF THE INVENTION

The invention provides a composition (e.g., a therapeutic composition), which may be in the form of a powder, e.g. a dry powder, comprising chitosan particles. The composition of the invention has been found to exhibit therapeutic efficacy in treating or preventing a wide range of oral, e.g., dental, conditions including infection, inflammation, gingivitis, periodontitis, surgical wounds, traumatic injuries to oral tissue, bone loss, e.g., associated with dental disease, and biofilms. It is believe that the chitosan used in the composition of the invention may have efficacy, for example, as an anti-infective, e.g., an antibacterial agent, an anti-biofilm agent (e.g., for breaking up and/or for preventing the formation of biofilms), and/or an anti-inflammatory agent, and may provide other therapeutic benefits as well such, e.g., as a local anesthetic, an antiseptic, an analgesic, or a combination thereof. The composition of the invention may consist essentially or primarily of the chitosan particles as described herein, e.g., wherein the components of the composition, other than the chitosan, may include residual water, impurities or by-products that may inherently exist in the chitosan, and the like. Alternatively, the composition of the invention may further include one or more additional components, e.g., a pharmaceutically acceptable carrier, excipient, and/or additional active agent (e.g., analgesic, antiseptic, anti-inflammatory agent, antibacterial agent, etc.), and the like, in combination with chitosan particles as described herein. In some embodiments, the composition of the invention consists essentially of the chitosan particles as described herein. Thus, the composition of the invention may consist primarily, if not exclusively, of the chitosan particles as the primary component and sole active agent. In other embodiments, the composition of the invention comprises chitosan particles as described herein and a pharmaceutically acceptable carrier, excipient, and/or additional active agent, and/or the like, as described herein.

In some embodiments, the composition of the invention is in the form of a powder. The powder may contain less than 20 wt. % water, for example, less than 10 wt. % water, less than 8 wt. % water, less than 7 wt. % water, less than 6 wt. % water, less than 5 wt. % water, less than 4 wt. % water, less than 3 wt. % water, less than 2 wt. % water, less than 1 wt. % water, less than 0.5 wt. % water, or less than 0.1 wt. % water. In some embodiments, the powder comprises less than 5 wt. % water, less than 1 wt. % water, or less than 0.1 wt. % water. The powder may be in the form of a non-flowing powder (e.g., a non-flowing dry powder), a free-flowing powder (e.g., a free-flowing dry powder), and/or a compactible powder (e.g., a compactible dry powder). In some embodiments, the composition of the invention consists essentially of the chitosan particles as described herein, and is in the form of a powder, e.g., in the form of a dry powder, as described herein.

In some embodiments, the composition of the invention, e.g., a composition that consists essentially of the chitosan particles as described herein, may contain up to about 30 wt. % water. For example, a composition that consists essentially of the chitosan particles as described herein may comprise from about 1 wt. % to about 30 wt. %, from about 5 wt. % to about 30 wt. %, or from about 10 wt. % to about 30 wt. % water. In some embodiments, it may be advantageous to adjust the water content of the composition (up or down) to achieve physical properties, compaction properties, adhesion properties, etc., that may be desirable for a particular application. The powder may be in the form of a non-flowing powder, a free-flowing powder, and/or a compactible powder. In some embodiments, it may be advantageous to formulate the composition into a paste.

The chitosan in the composition of the invention may have any suitable degree of deacetylation relative to chitin. For example, the chitosan may have a degree of deacetylation of at least about 50% relative to chitin, a degree of deacetylation of at least about 60% relative to chitin, a degree of deacetylation of at least about 70% relative to chitin, a degree of deacetylation of at least about 75% relative to chitin, a degree of deacetylation of at least about 80% relative to chitin, or a degree of deacetylation of at least about 85% relative to chitin. In some embodiments, the chitosan in the composition of the invention has a degree of deacetylation of from about 50% to about 100% relative to chitin, a degree of deacetylation of from about 80% to about 100% relative to chitin, a degree of deacetylation of from about 85% to about 100% relative to chitin, a degree of deacetylation of from about 50% to about 99% relative to chitin, a degree of deacetylation of from about 80% to about 99% relative to chitin, or a degree of deacetylation of from about 85% to about 99% relative to chitin. In certain embodiments, the chitosan has a degree of deacetylation of at least about 85% relative to chitin.

The chitosan in the composition of the invention may have any suitable weight average molecular weight. In some embodiments, the chitosan has a weight average molecular weight from about 1 kDa to about 500 kDa. For example, the chitosan may have a weight average molecular weight from about 1 kDa to about 400 kDa, from about 1 kDa to about 300 kDa, from about 1 kDa to about 200 kDa, from about 1 kDa to about 100 kDa, from about 1 kDa to about 50 kDa, from about 1 kDa to about 25 kDa, from about 3 kDa to about 25 kDa, or from about 4 kDa to about 20 kDa. In certain embodiments, the chitosan has a weight average molecular weight from about 4 kDa to about 20 kDa.

The chitosan in the composition of the invention may be unsubstituted or substituted such that the chitosan may be in the form of a derivative thereof. For example, the chitosan may be modified by acylation (e.g., O-acylation), carboxylation, alkylation (e.g., N-alkylation or O-alkylation), and quaternization (e.g., quaternary ammonium chitosan). In some embodiments, the chitosan may be esterified, sulfated, sulfonated, carboxymethylated, alkylated, sulfonylated, alkanoylated, crosslinked, or any combination thereof. For example, the chitosan may be O-carboxymethyl chitosan, N-carboxymethyl chitosan, N,N,N-trimethyl chitosan, N-2-hydroxypropyl trimethyl ammonium chloride chitosan, N-trimethylaminoethylmethacrylate chitosan, methoxypolyethylene glycol and carboxymethyl chitosan, ammonium N-alkyl chitosan, quaternized N-alkyl chitosan, quaternized N-aryl chitosan, O-imidazolyl quaternary ammonium chitosan, glutaraldehyde cross-linked chitosan, trimethyl chitosan, hydroxypropyl chitosan, hydroxybutyl chitosan, or hydroxyethyl chitosan. The chitosan in the composition of the invention may be modified or further modified to include a fatty chain (e.g., by way of a fatty acid or the like). In some embodiments, the chitosan may be esterified with one or more acids (e.g., lactic acid, hyaluronic acid, etc.).

The chitosan in the composition of the invention, if substituted, may have any suitable degree of substitution. For example, up to 90% (e.g., up to 80%, up to 70%, up to 60%, up to 50%, up to 40%, up to 30%, 5% to 90%, 5% to 80%, 5% to 70%, 5% to 60%, up to 50%, up to 40%, 10% to 90%, 10% to 80%, 10% to 70%, 10% to 60%, 10% to 50%, 10% to 40%, 20% to 90%, 20% to 80%, 20% to 70%, 20% to 60%, 20% to 50%, or 20% to 40%) of the substitutable oxygen atoms of the chitosan may be substituted. Alternatively, or additionally, up to 90% (e.g., up to 80%, up to 70%, up to 60%, up to 50%, up to 40%, up to 30%, 5% to 90%, 5% to 80%, 5% to 70%, 5% to 60%, up to 50%, up to 40%, 10% to 90%, 10% to 80%, 10% to 70%, 10% to 60%, 10% to 50%, 10% to 40%, 20% to 90%, 20% to 80%, 20% to 70%, 20% to 60%, 20% to 50%, or 20% to 40%) of the substitutable nitrogen atoms may be substituted.

The chitosan particles in the composition of the invention may have any suitable particle size distribution. In some embodiments, the chitosan particles may have a particle size distribution with a mode of about 0.1 μm to about 100 μm, e.g., from about 1 μm to about 100 μm (e.g., a mode of about 1 μm to about 50 μm, a mode of about 1 μm to about 20 μm, a mode of about 1 μm to about 10 μm, a mode of about 1 μm to about 8 μm, a mode of about 1 μm to about 6 μm, a mode of about 2 μm to about 10 μm, a mode of about 2 μm to about 8 μm, a mode of about 2 μm to about 6 μm, or a mode of about 3 μm to about 5 μm). In some embodiments, the chitosan particles in the composition of the invention may have a particle size distribution with a mode of about 3 μm to about 5 μm. Alternatively, or additionally, the chitosan particles in the composition of the invention may have (a) a d10 of (i.e., wherein 10% of particles are smaller than) at most about 10 μm, for example, a d10 of at most about 8 μm, a d10 of at most about 7 μm, a d10 of at most about 6 μm, a d10 of at most about 5 μm, a d10 of at most about 4 μm, or a d10 of at most about 3 μm, (b) a d50 of (i.e., wherein 50% of particles are smaller than) at most about 20 μm, for example, a d50 of at most about 10 μm, a d50 of at most about 8 μm, a d50 of at most about 7 μm, a d50 of at most about 6 μm, or a d50 of at most about 5 μm, and/or (c) a d90 of (i.e., wherein 90% of particles are smaller than) at most about 30 μm, for example, a d90 of at most about 25 μm, a d90 of at most about 20 μm, a d90 of at most about 15 μm, or a d90 of at most about 10 μm.

The chitosan particles in the composition of the invention may be prepared by any suitable method. For example, the chitosan particles may be obtained by spray drying, ball milling, jet milling, cryo-milling, microfluidization, lyophilization, or a combination thereof. In some embodiments, the chitosan particles are obtained by spray drying. The chitosan particles may be further ground, pulverized, filtered, milled, or sifted, etc. to achieve a suitable particle size distribution.

In some embodiments, the chitosan in the composition of the invention may be in the form of a pharmaceutically acceptable salt (e.g., a pharmaceutically acceptable acid addition salt). The phrase “pharmaceutically acceptable salt” herein refers to salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts that may be suitable for purposes of the invention are known in the art. For example, S. M. Berge et al. describe examples of suitable pharmaceutically acceptable salts in J. Pharmaceutical Sciences, 66:1 et seq (1977). For example, for acid addition salts, the nitrogen atoms of the chitosan molecule may form salts with acids. Examples of suitable acids for salt formation may include, e.g., hydrochloric, hyaluronic, sulfuric, phosphoric, acetic, citric, oxalic, malonic, salicylic, malic, fumaric, succinic, ascorbic, maleic, methanesulfonic, and other acids that are known to those of ordinary skill in the art. In some embodiments, the pharmaceutically acceptable salt may be formed from an amino acid (e.g., lysine and arginine). The salts may be prepared by contacting the free base form of the chitosan with a sufficient amount of the desired acid to produce a salt in a conventional manner. The chitosan also may be in the form of a free base. The free base forms may be regenerated by treating an acid addition salt with a suitable dilute aqueous base solution such as a dilute aqueous hydroxide solution (e.g., sodium hydroxide, potassium hydroxide, etc.), a suitable carbonate salt, e.g., potassium carbonate, a suitable amine, e.g., ammonia, and/or a suitable bicarbonate salt, e.g., sodium bicarbonate. The free base forms may differ from their respective salt forms in certain physical properties, such as solubility in (or affinity to) polar solvents, although the acid salts and their respective free base forms are believed to be suitable for purposes of formulating, administering, and/or treating oral conditions in accordance with the invention.

In some embodiments, the chitosan may be in the form of a pharmaceutically acceptable acid addition salt. For example, the chitosan may be in the form of an acid addition salt such as, e.g., hydrochloride, hydrobromide, hyaluronate, sulfate, phosphate, nitrate, formate, acetate, lactate, succinate, citrate, tartrate, or ascorbate. In certain embodiments, the chitosan is in the form of a hydrochloride salt.

The composition of the invention may be in the form of a powder, e.g., a dry powder, which is preferably compactible, e.g., into a pellet, e.g., which may be inserted directly into the periodontal pocket. The powder also may be formulated into, e.g., a paste or a bioadhesive gel. The composition of the invention also may form a pellet, a paste, or a bioadhesive gel when contacted with an aqueous fluid, e.g., at the site of application in the oral cavity, e.g., inside or near a periodontal pocket. For example, the composition of the invention (e.g., in the form of a powder) comprising chitosan particles may form a pellet, a paste, or a bioadhesive gel following contact with the aqueous environment of an oral wound or tissue, for example, following application into a periodontal pocket, or if contacted with water or aqueous fluid prior to application to an oral wound or tissue.

The composition of the invention, e.g., in the form of a powder comprising chitosan particles, may further comprise one or more additional active agents. For example, the composition of the invention, e.g., in the form of a powder comprising chitosan particles, may further comprise a therapeutically effective amount of an additional active agent which may be a local anesthetic, an antiseptic, an analgesic, an anti-inflammatory agent, an antibacterial agent, an anti-infective agent, and the like, or a combination thereof.

In some embodiments, the composition of the invention, e.g., in the form of a powder comprising chitosan particles, may further comprise an antibacterial agent. The antibacterial agent may be any suitable compound that aids in the prevention and/or elimination of bacteria that may be associated with oral or dental disease. For example, the antibacterial agent may be effective in preventing and/or treating an infection by one or more of Porphyromonas gingivalis, Prevotella intermedia, Fusobacterium nucleatum, Eikenella corrodens, and Actinobacillus actinomycetemcomitans. In some embodiments, the antibacterial agent may comprise a tetracycline such as, for example, minocycline or a pharmaceutically acceptable salt thereof. In certain embodiments, the composition of the invention may further comprise minocycline hydrochloride.

In some embodiments, the composition of the invention, e.g., in the form of a powder which comprises chitosan particles, may further include one or more additional components such as, e.g., a pharmaceutically acceptable carrier. The carrier may include a component that functions as a binder, a glidant, a dispersant, a disintegrant, an excipient, a lubricant, or a combination thereof. Alternatively, or additionally, the carrier may comprise a buffer, a preservative, a flavor enhancer, a coloring agent, or a combination thereof. In some embodiments, the carrier may include a binder, a glidant, a dispersant, a disintegrant, an excipient, a lubricant, or a combination thereof. Suitable binders may include, e.g., microcrystalline cellulose, gum tragacanth, gelatin, or a combination thereof. The dispersant may comprise, e.g., a surfactant; the disintegrant may comprises alginic acid, Primogel, corn starch, or a combination thereof; the excipient may comprise, e.g., a filler, starch, lactose, or a combination thereof; the lubricant may comprise, e.g., magnesium stearate; and the glidant may comprise, e.g., colloidal silicon dioxide.

The composition of the invention may further include one or more preservatives such as, for example, sorbic acid or a salt thereof (e.g., sodium sorbate, potassium sorbate), benzoic acid or a salt thereof (e.g., sodium benzoate, potassium benzoate), one or more sulfites (e.g., sodium sulfite) and/or bisulfites (e.g., sodium metabisulfite), one or more nitrites (e.g., sodium nitrite), ascorbic acid or a salt thereof, or a combination thereof.

In some embodiments, compositions of the invention which contain an additional active agent (i.e., in addition to the chitosan) as described herein are preferably capable of releasing the additional active agent into an oral wound of a patient (e.g., a human) to promote a desired efficacy of the additional active agent. For example, the chitosan particles may release the additional active agent into the periodontal pocket of the patient (e.g., a human) to facilitate or accelerate treatment of the oral condition.

In some embodiments, the composition consists essentially of the chitosan particles as described herein in the form of a powder (e.g., a dry powder). For example, the composition may be in the form a powder containing chitosan particles as described herein, water (e.g., less than 1 wt. % water, less than 0.5 wt. % water, or less than 0.1 wt. % water), and less than about 1 wt. %, less than about 0.1 wt. %, or less than about 0.01 wt. % of any other components, i.e., any components other than the chitosan and water. In some embodiments, the composition may be in the form of a powder containing the chitosan particles as described herein, water (e.g., less than 1 wt. % water, less than 0.5 wt. % water, or less than 0.1 wt. % water), and less than 0.001 wt. % or less than 0.0001 wt. % of other components, i.e., any components other than the chitosan and water.

The invention also provides a method for treating or preventing an oral condition (e.g., inflammation, bleeding, suppuration, gum recession, bone loss, tooth decay, biofilms, sores, injuries, lesions, and ulcers of the oral mucosa) in a patient, the method comprising administering to the patient a therapeutically effective amount of the composition of the invention, to treat the oral condition. In some embodiments, the oral condition includes gingivitis, periodontitis, peri-implantitis, peri-implant mucositis, oral bone loss, biofilms (e.g., by breaking up and/or inhibiting biofilm formation), and combinations thereof. For example, the composition of the invention, e.g., in the form of a powder comprising chitosan particles as described herein, may be used in the treatment of periodontitis, peri-implantitis, peri-implant mucositis, biofilms (prevention or disintegration), bone loss, or a combination thereof. In certain embodiments, the oral condition (e.g., an oral condition associated with periodontitis, peri-implantitis, peri-implant mucositis, or a combination thereof) may include one or more of infection, inflammation, bleeding, suppuration, gum recession, tooth decay, pocket depth increase, bone loss, biofilms, or a combination thereof.

In some embodiments, the method comprises subgingivally administering to the patient a therapeutically effective amount of the composition of the invention to treat the condition in the patient. In certain embodiments, the method comprises subgingivally administering to the patient a therapeutically effective amount of the composition of the invention may comprise inserting the composition, e.g., as a powder or pellet (e.g., compacted pellet) into a periodontal pocket of the patient, to treat the condition in the patent. The composition (e.g., administered as a powder comprising chitosan particles as described herein) may be retained in the oral cavity (e.g., within a periodontal pocket, as a wound dressing or as part of a wound dressing, or inside of a wound, e.g., within a sutured wound) of a patent for any suitable amount of time. For example, the composition may be retained in the oral cavity (e.g., within a periodontal pocket, as a wound dressing or as part of a wound dressing, or inside of a wound, e.g., within a sutured wound) of the patient for at least about 12 hours, at least about 24 hours, at least about 2 days, at least about 3 days, at least about 7 days, or at least about 14 days. In some embodiments, the composition may be retained in the oral cavity (e.g., within a periodontal pocket, as a wound dressing or as part of a wound dressing, or inside of a wound, e.g., within a sutured wound) of the patient for up to about 12 hours (e.g., about 1-12 hours), up to about 24 hours, up to about 2 days (e.g., about 1-2 days), up to about 3 days (e.g., about 1-3 days), up to about 7 days (e.g., about 1-7 days), or up to about 14 days (e.g., about 1-14 days, about 7-14 days, etc.).

In some embodiments, the method may include topically administering the composition to a tissue within the oral cavity of the patient. For example, the composition may be topically applied to any tissue surface, e.g., gum tissue, periodontal tissue, buccal tissue, mucosal tissue, etc., within the oral cavity of the patient, e.g., with a spatula. In some embodiments, the composition of the invention may be administered, e.g., topically, as a wound dressing by itself or as an active component of a wound dressing, for example, as a wound dressing for diseased or wounded tissue in the oral cavity (e.g., gum tissue, periodontal tissue, buccal tissue, mucosal tissue, etc.,).

In some embodiments, the method comprises sub-dermally or sub-surgically administering the composition within an oral wound of the patient. For example, the composition may be applied inside of an oral wound (e.g., during or after a surgical procedure) prior to suturing such that, after suturing, the composition is retained within the oral wound, e.g., beneath the surface tissues and inside of the sutured wound.

The composition of the invention also may be administered as described herein, e.g., as an adjunct to a scaling or a root planing procedure, periodontal flap surgery, gingival graft, implant placement, bone graft, or an extraction procedure. The composition has been found to be surprisingly efficacious in treating a broad range of oral conditions such as, for example, infection, inflammation, bleeding, suppuration, gum recession, tooth decay, biofilms, bone loss, sores, lesions, traumatic injuries, and ulcers.

A therapeutically effective amount of the composition of the invention may include any amount that is minimally needed to treat (e.g., prevent or reduce symptoms associated with) an oral condition. An effective amount may include an amount of the composition effective to form a barrier on the surface of the wound that is sufficiently protective to alleviate pain and/or promote wound healing. For example, the therapeutically effective amount of the composition may comprise at least about 0.01 mg of the chitosan particles, at least about 0.1 mg of the chitosan particles, at least about 1 mg of the chitosan particles, at least about 5 mg of the chitosan particles, at least about 10 mg of the chitosan particles, at least about 50 mg of the chitosan particles, at least about 100 mg of the chitosan particles, or at least about 500 mg of the chitosan particles. In some embodiments, the therapeutically effective amount of the composition comprises from about 0.01 mg to about 1 g of the chitosan particles, from about 0.01 mg to about 500 mg of the chitosan particles, or from about 0.01 mg to about 100 mg of the chitosan particles. In certain embodiments, administering a therapeutically effective amount includes applying from about 0.1 mg to about 10 mg, e.g., from about 0.1 mg to about 5 mg, from about 0.5 mg to about 5 mg, from about 0.5 mg to about 4 mg, from about 0.5 mg to about 3 mg, from about 0.5 mg to about 2 mg, from about 0.5 mg to about to about 1.5 mg, e.g., 1 mg, of the composition of the invention, e.g., a composition that consists essentially of the chitosan particles as described herein, e.g., in the form of a powder, to a tissue of the oral cavity of a patient to treat the condition.

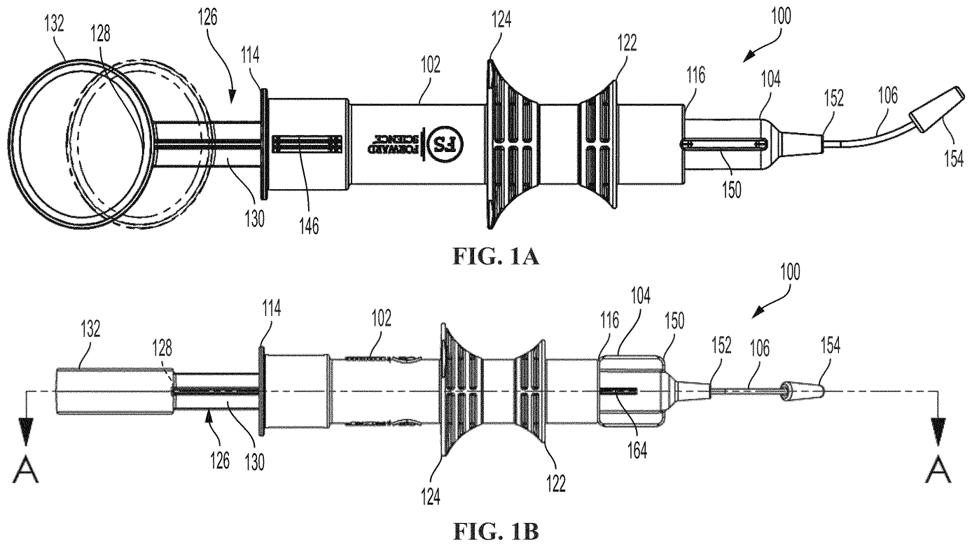

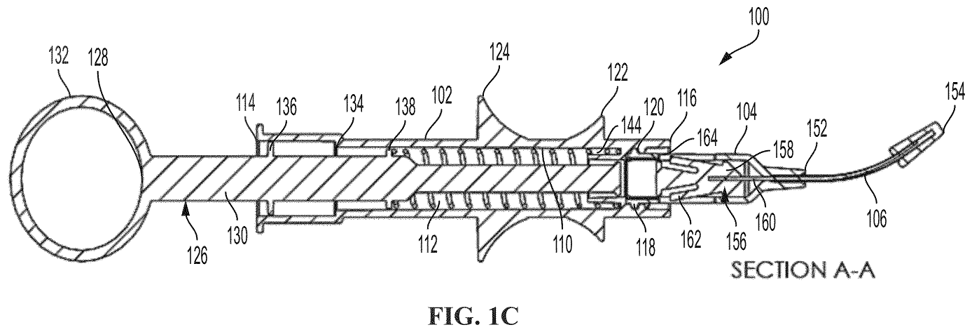

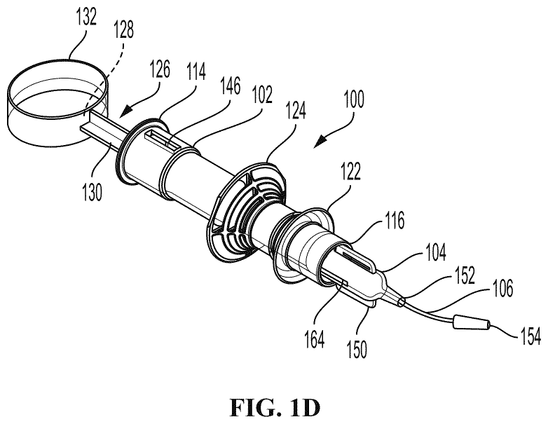

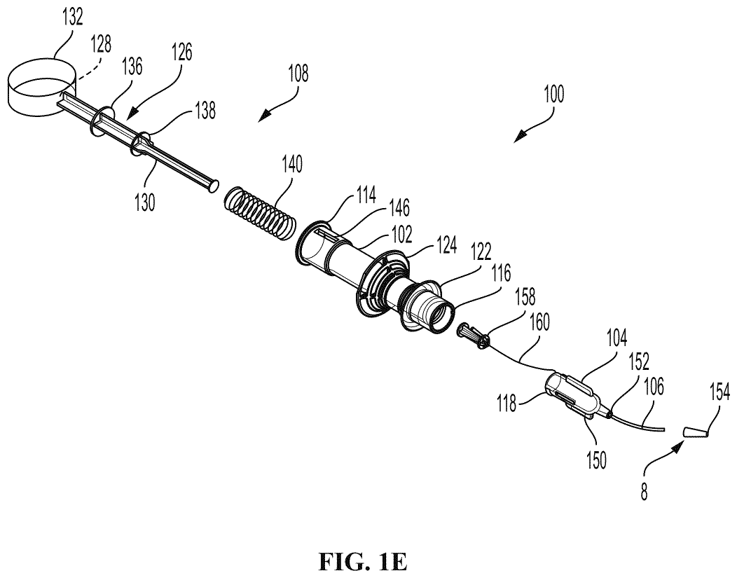

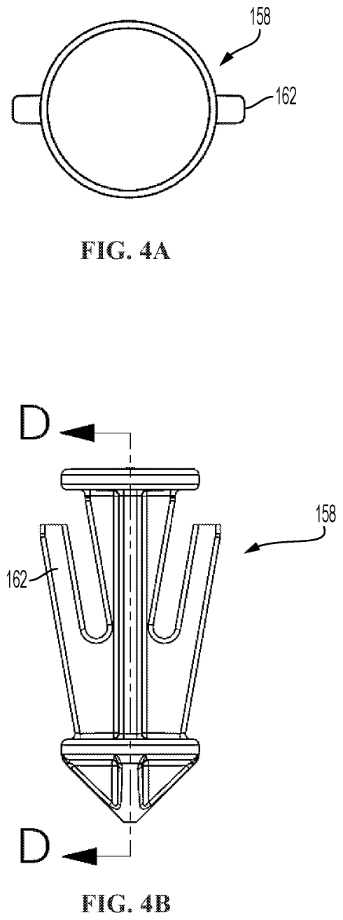

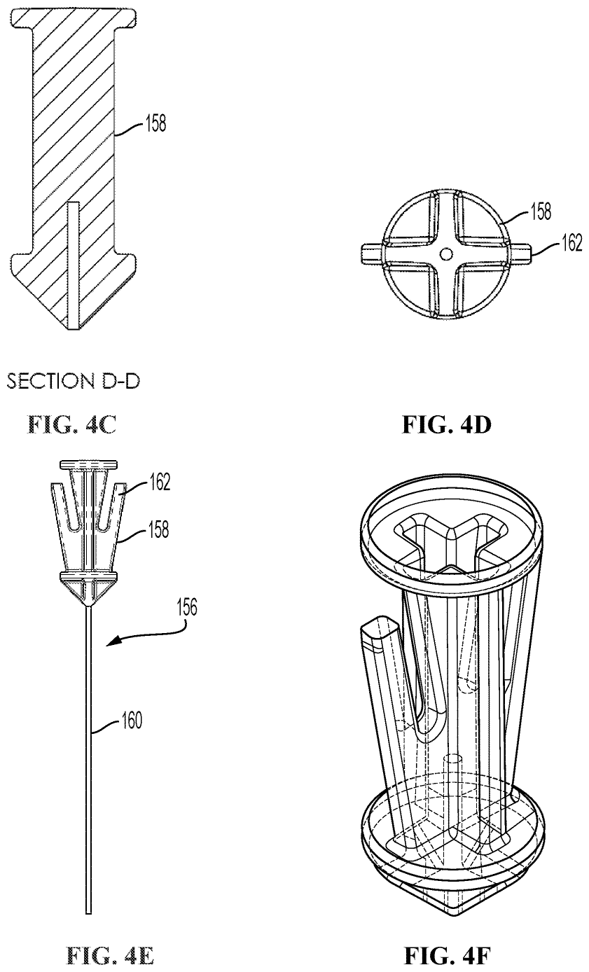

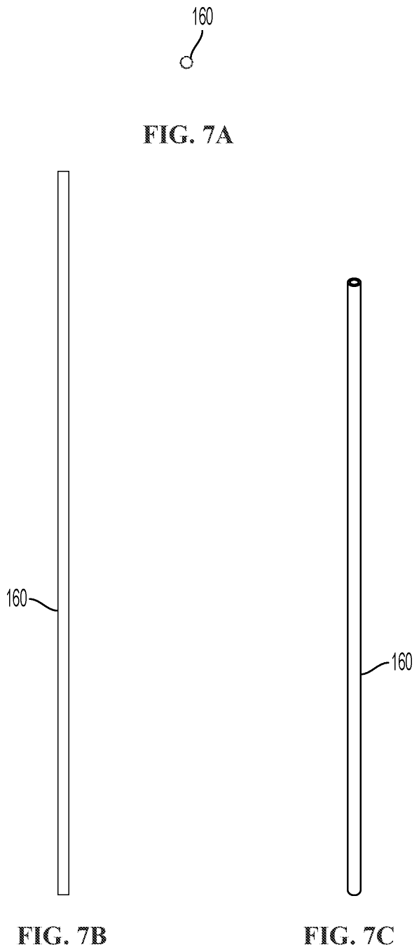

The composition of the invention, e.g., a composition that consists essentially of chitosan particles in the form of a powder as described herein, or, e.g., a composition comprising chitosan particles as described herein generally, may be applied to a wound in the oral cavity using any suitable method. For example, the composition of the invention may be applied directly to the wound surface with a syringe applicator or by other means, e.g., manually. In some embodiments, the composition of the invention is administered by way of a unit dose cartridge comprising a stainless needle attached to a plastic tip (e.g., a ridged tip) or metal tip, wherein the tip is inserted into the periodontal pocket or wound and the composition is expelled through the tip and into the periodontal pocket or wound to administer the composition, e.g., as a powder or pellet. In other embodiments, the composition of the invention is administered by way of a unit dose cartridge comprising a deformable tip or a non-ridged tip, wherein the deformable tip or non-ridged tip is inserted into the periodontal pocket and the powder is expelled through the tip and into the periodontal pocket to administer the composition, e.g., as a powder or pellet. In some embodiments, the composition may be administered, e.g., in the form of a powder, e.g., a powder comprising chitosan particles as described herein, or a powder that consists essentially of chitosan particles as described herein, by way of a multi dose cartridge comprising a stainless needle attached to a plastic tip (e.g., a ridged tip) or metal tip, wherein the tip is inserted into the periodontal pocket or wound and the composition is expelled through the tip and into the periodontal pocket or wound to administer the composition. In other embodiments, the composition, e.g., in the form of a powder, may be administered by way of a multi dose cartridge comprising a deformable tip or a non-ridged tip, wherein the deformable tip or non-ridged tip is inserted into the periodontal pocket and the composition is expelled through the tip and into the periodontal pocket to administer the composition. In certain embodiments, the unit dose or multi dose cartridge is a disposable unit dose or multi dose cartridge. For example, the composition of the invention may be applied directly to the wound surface with an applicator comprising any combination of features shown in FIGS. 1-8 and/or as otherwise described herein.

In some embodiments, the composition of the invention, e.g., in the form of a powder comprising or consisting essentially of chitosan particles as described herein, may be applied to an oral cavity by compacting the composition into a pellet and applying the pellet to a tissue, e.g., by inserting the pellet into a periodontal pocket, in the oral cavity of the patient. In other embodiments, the composition of the invention, e.g., in the form of a powder comprising or consisting essentially of chitosan particles as described herein, may be applied as a paste, e.g., using a spatula or other suitable applicator, to a tissue within the oral cavity of the patient.

The invention further provides an applicator 100 comprising any combination of features shown in FIGS. 1-8 and/or as otherwise described herein. The specific structure and relative dimensions provided in the figures are exemplary dimensions of at least one embodiment of the applicator 100 and/or components thereof. Other dimensions are envisioned within the scope of this disclosure.

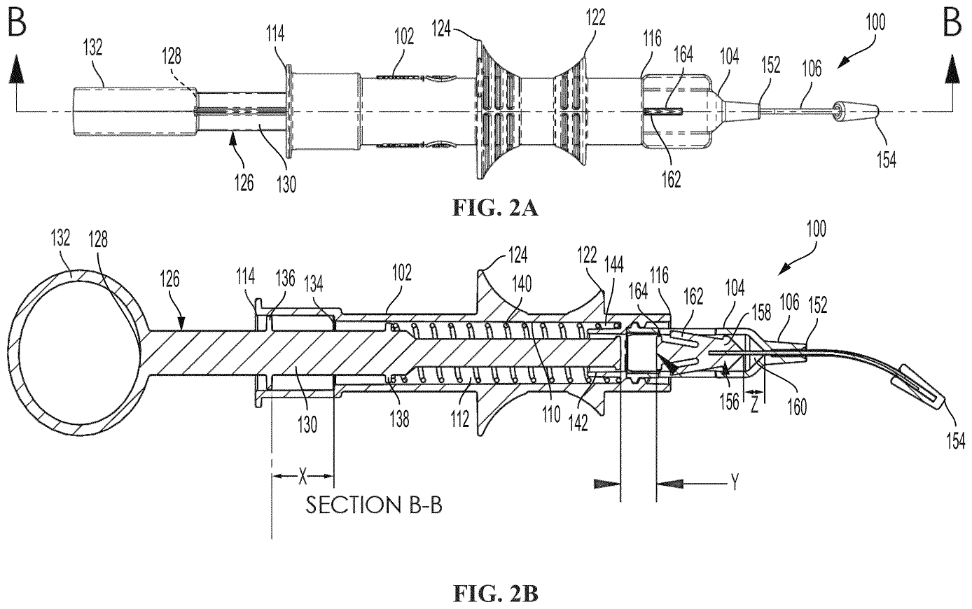

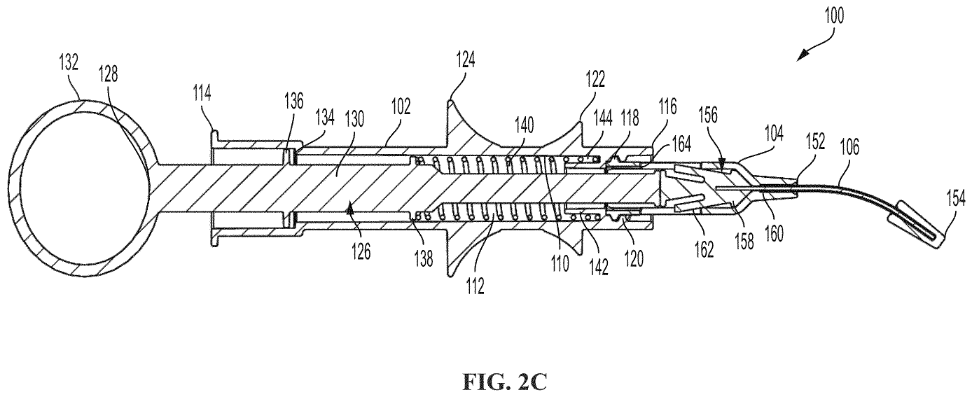

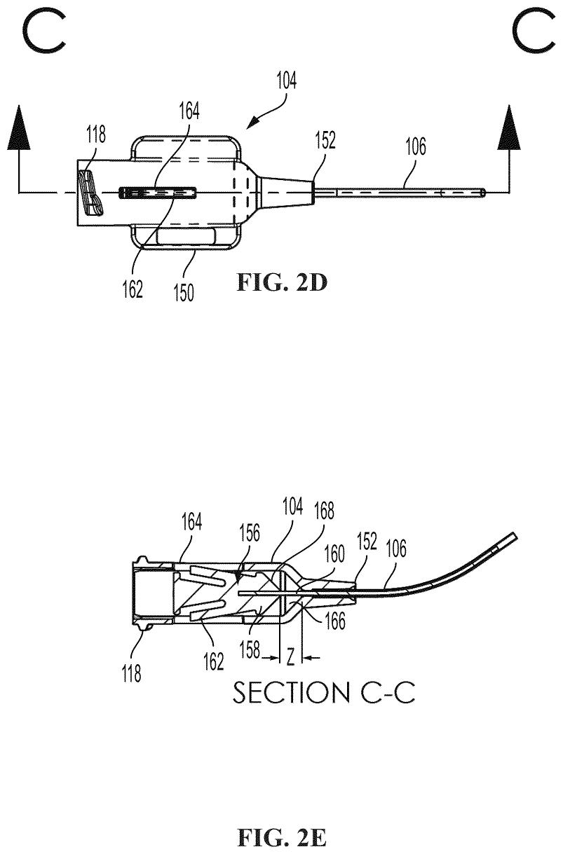

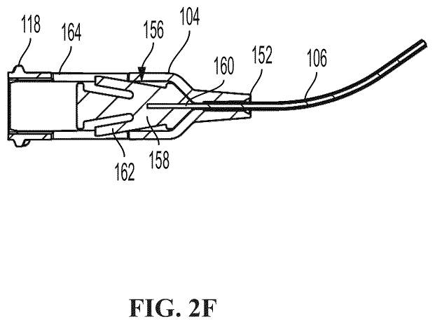

In some embodiments, the invention provides an applicator 100 for administration of a medicament such as a hydrogel or powder to a surface. An exemplary embodiment of the applicator 100 is illustrated in FIGS. 1A-2C. The applicator 100 includes a barrel 102, to which a tip 104 having a delivery needle 106 is coupled. An actuation assembly 108 is provided to facilitate delivery of the medicament through the delivery needle/conduit 106.

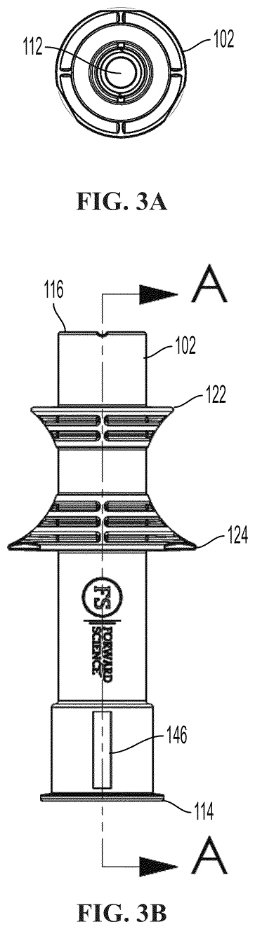

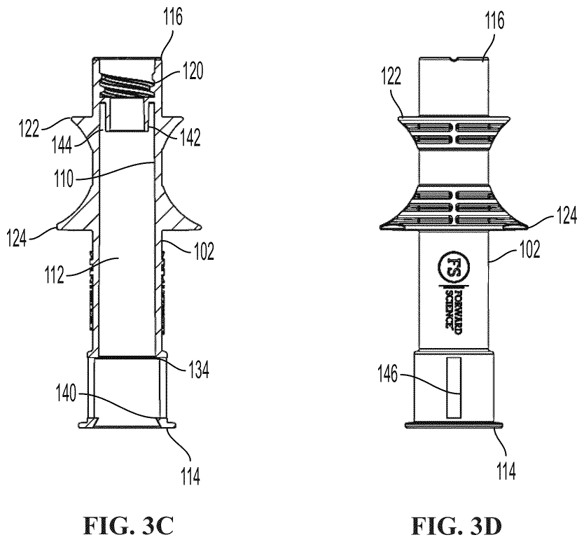

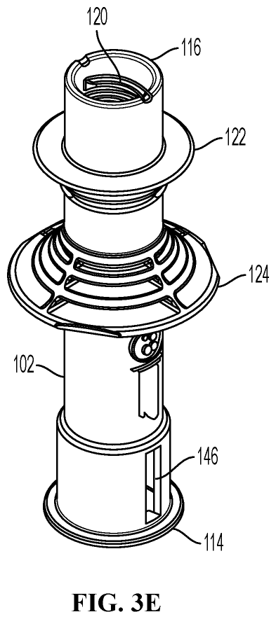

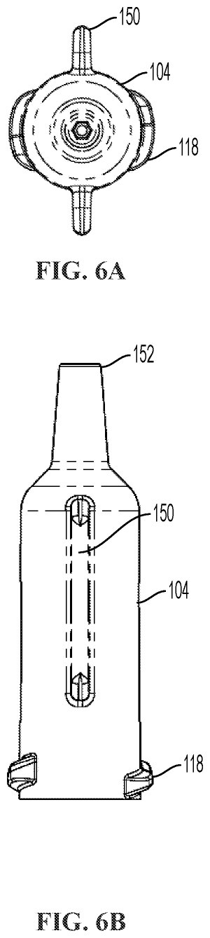

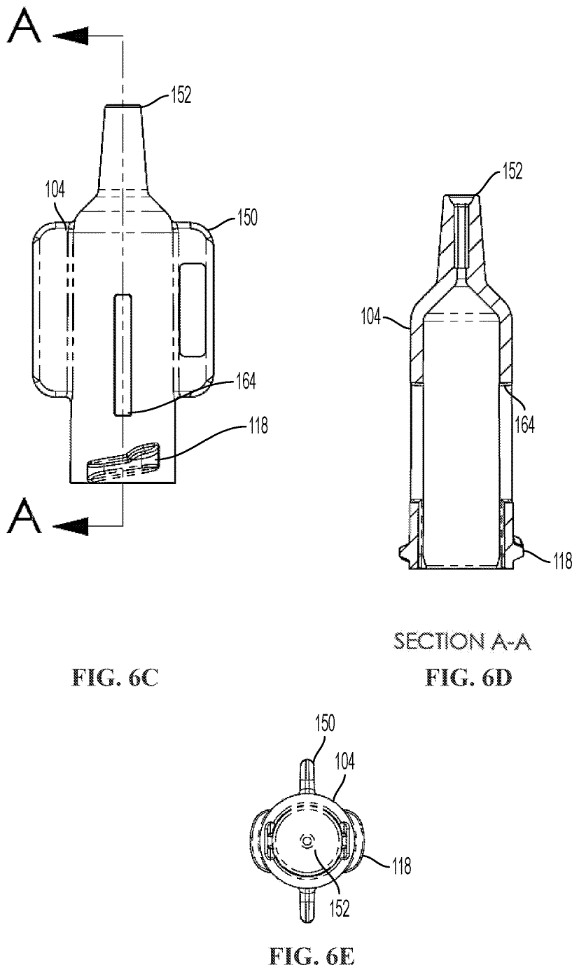

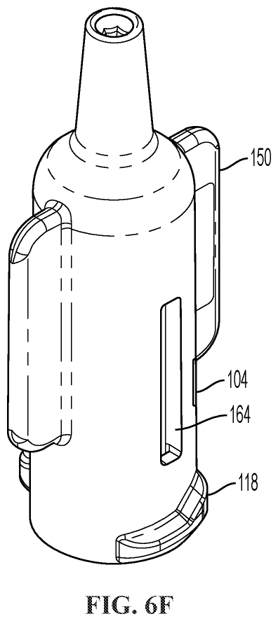

The barrel 102 may be generally cylindrical with an internal surface 110 that forms a longitudinally extending internal chamber 112 that is open at a proximal end 114 to receive the actuation assembly 108, and at a distal end 116 for coupling with the tip 104. An exemplary embodiment of the barrel 102 is shown in detail in FIGS. 3A-3E, while an exemplary embodiment of the tip 104 is shown in detail in FIGS. 6A-6F.

The tip 104 may be coupled to the distal tip 114 of the barrel 102 by any appropriate arrangement. In the embodiment illustrated in FIGS. 1A-3E and 6A-F, the tip 104 is coupled to the barrel 102 by threads 118, 120 on the tip 104 and barrel 102, respectively. Other arrangements consistent with the teachings of this disclosure, such as an interference or snap connection, also may be used.

An outer surface of the barrel 102 may include one or more engagement surfaces or flanges to provide an operator gripping surfaces for use when assembling the tip 104 to the barrel 102 or when administering a medicament. For example, a generally radially extending flange 122 may be utilized in assembling the tip 104 to the barrel 102, while generally radially extending flange 124 may be utilized when administering a medicament.

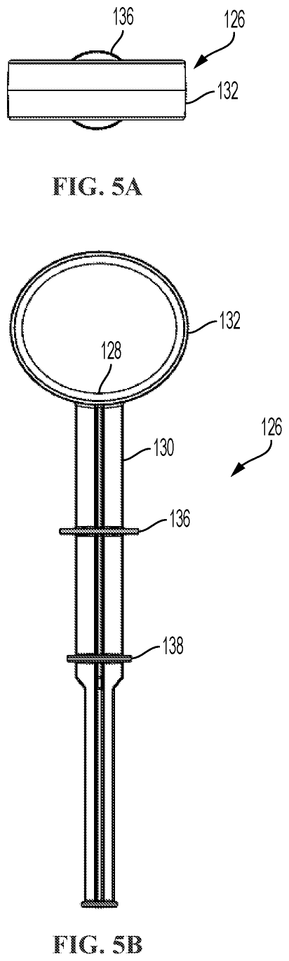

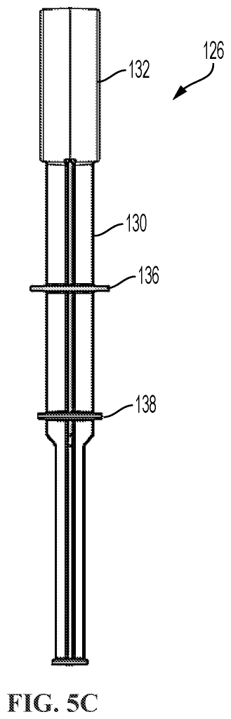

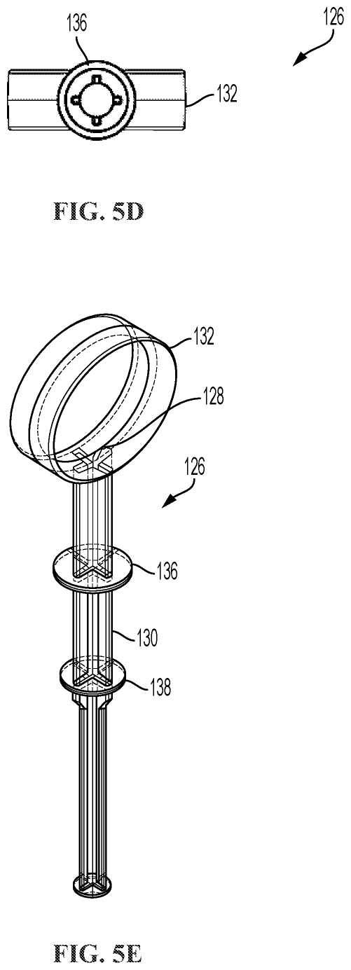

In order to deliver a medicament through the delivery needle/conduit 106, the actuation assembly 108 may include a drive stick 126. An exemplary embodiment of the drive stick/plunger 126 is illustrated in FIGS. 5A-5E. The drive stick/plunger 126 includes an engagement surface 128 and an elongated drive element 130 adapted to be received within the internal chamber 112 of the barrel 102. In the illustrated embodiment, the engagement surface 128 is provided within a generally annular element 132. In this way, an operator may, for example, place fingers on either side of the barrel 102 between the flanges 122, 124, and a thumb in the annular element to apply a force to advance the drive stick/plunger 126 within the barrel 102.

In at least one embodiment, the drive stick/plunger 126 and the barrel 102 may include structures that interfere with one another in order to control or limit the axial and/or radial movement of the drive stick/plunger 126 within the barrel 102. For example, the barrel 102 may include a flange or surface 134 extending radially inward from the internal surface 110, while the drive stick 126 may include a radially outwardly extending flange 136 configured to engage the surface 134 of the barrel 102 in order to limit axial travel of the drive stick 126 within the longitudinally extending internal chamber 112 of the barrel 102. It will be appreciated that the flange 136 of the drive stick/plunger 126 and the internal surface 110 of the barrel 102 may be configured to control radial movement of the drive stick 126 within the longitudinally extending internal chamber 112 in order to facilitate accurate axial movement of the drive stick 126 relative to the barrel 102. Similarly, the drive stick 126 may be provided with one or more additional radially extending surfaces or flanges 138 that center or align the drive stick 126 along the drive axis within the barrel 102.

In order to maintain the drive stick 126 within the barrel 102, the barrel 102 may further include an interference structure, such as a flange 140 extending radially inward (see FIG. 3C). In this way, the flange 140 may be flexible enough to permit assembly of the drive stick 126 into the barrel 102, yet rigid enough to limit outward movement of the drive stick 126 relative to the barrel 102. Those of skill in the art will appreciate that alternative mechanisms may be provided for maintaining the drive stick 126 within the barrel 102.

The actuation assembly 108 may additionally include a biasing element 142, such as a spring, that may be positioned to bias the drive stick 126 in a given position, here, in the unactuated position illustrated in FIG. 1C. While the illustrated embodiment includes a coil spring, those of skill in the art will appreciate that an alternative biasing element may be provided.

In order to maintain the biasing element 140 in position within the barrel 102, the barrel 102 may provide limiting structures. In the illustrated embodiment, the barrel 102 includes an axially extending, generally cylindrical flange element 142, here, within the internal chamber 112. In this way, a distal end of the biasing element 140 is disposed within a generally cylindrical chamber 144 formed between the flange element 142 and the internal wall 110 of the barrel 102. While the flange element 142 is illustrated as a continuous cylindrical element, those of skill in the art will appreciate that the flange element 142 may include one or more breaks or may be of a different design. Further, those of skill in the art will appreciate that the biasing element 140 may be held in position by an alternative or additional arrangement. In order for the further retain the biasing element 140 and to provide movement against the bias, the radially extending flange 138 may be sized to bear against the proximal end of the biasing element 136.

In order to allow an operator to ready discern the position of the drive stick/plunger 126 within the barrel 102, the barrel 102 may be provided with a viewing window 146. In this way, an operator may view the position of the flange 136 through the viewing window 146 in order to discern whether the applicator 100 is in an actuated or unactuated position.

The tip 104 is coupled to the distal end 116 of the barrel 102. The tip 104 may be a generally cylindrical structure with a closed distal end 152 from which the needle/conduit 106 extends. While the needle/conduit 106 may be fabricated of any appropriate material, in at least one embodiment, the needle/conduit 106 is fabricated of metal.

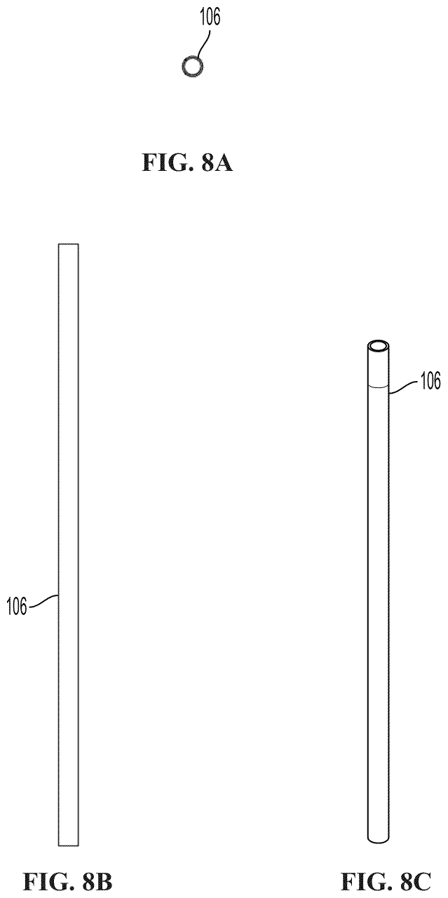

In at least one embodiment, the outer diameter of the needle/conduit 106 is on the order of 0.042 inches (1.07 mm). In some embodiments, the outer diameter of the needle/conduit 106 is less than 1.4 mm, less than 1.3 mm, less than 1.2 mm, or less than 1.1 mm. In other embodiments, however, the outer diameter is on the order of up to 0.035 inches (0.9 mm). In other embodiments, the distal end of the needle/conduit 106 has an outer diameter of from about 0.025 inches (0.63 mm) to about 0.05 inches (1.27 mm), e.g., from about 0.025 inches (0.63 mm) to about 0.048 inches (1.22 mm), from about 0.025 inches (0.63 mm) to about 0.046 inches (1.17 mm), or about 0.025 inches (0.63 mm) to about 0.044 inches (1.12 mm), [etc.]. Moreover, while the needle/conduit 106 of FIGS. 8A-8C is illustrated as having a round cross-section, those of skill in the art will appreciate that the needle/conduit 106 could have an alternative cross-section, such as an oval cross-section, for example. In at least one embodiment, the distal end of the needle/conduit 106 is of a size that is the same as, similar to or smaller than tools generally utilized in a target area. Accordingly, the applicator 100 may be readily utilized without adjustment or filling by an operator.

A shield 154 may be provided to cover the end on the needle/conduit 106 in order to prevent an inadvertent contact or contamination with the tip of the needle/conduit 106, and to inhibit inadvertent dispensing of the medicament contained within the needle/conduit 106. While the tip 104 is received within the internal chamber 112 of the barrel 102, those of skill in the art will appreciate that an alternative embodiment may include a tip that is coupled to the exterior of the barrel 102, that is, the distal end 116 of the barrel 102 being at least partially received within the tip (not illustrated). In order to assist in assembly of the tip 104 with the barrel 102, the tip 104 may include radially extending arms 150.

The actuation assembly 108 further includes an actuator assembly 156 that is received within and axially movable within the tip 104. The actuator assembly 156 includes an actuator 158 from which a probe 160 extends. In assembly, the actuator assembly 156 is assembled into the tip 104 with the probe 160 extending within the needle/conduit 106, and the actuator 158 dispose within and axially moveable within the tip 104

In order to maintain the actuator assembly 156 within the tip 104 and to control the movement of the actuator assembly 156 within the tip 104, the actuator 158 includes one or more generally radially extending wings 162 that are biased to an outward position. In the illustrated embodiment a pair of radially extending wings 162 are provided, although it will be appreciated that three or more such wings 162 may be provided. The wings 162 are received in and axially movable within corresponding axially extending windows 164 in the tip 104. In assembly, the wings 162 may be flexed inward against the bias to assemble the actuator 158 into the proximal end of the tip 104 in an axial direction. When in position within the tip 104, the wings 162 return to their biased position within the windows 164. That is, the movement of the wings 162 to their outward position operates as an indication of the accurate positioning of the actuator 158 within the tip 104. The movement of the actuator assembly 156 in either the proximal or distal directions within the tip 104 is then limited by movement of the wings 162 within the windows 164. While the actuator 158 and the probe 160 may be formed of any appropriate material, in at least one embodiment the actuator 158 is formed of a polymeric material, and the probe 160 is formed of metal.

In at least one method of assembly, a medicament such as a powder, may be drawn into the interior of the needle/conduit 106 by way of a vacuum or the like. While a portion of the medicament within the needle/conduit 106 may be expelled as the probe 160 then is inserted into the interior of the needle/conduit 106, the outward flexing of the wings 162 as the actuator 158 is axially advanced into the tip 104 acts to provide an intermediate stop that results in the desired volume of medicament being retained within the needle/conduit 106 for application to a target site. In this way, it will appreciated that the relative lengths of the needle/conduit 106 and the length and/or positioning of the probe 160 within the needle/conduit 106 determine the volume of medicament retained.

Referring to FIGS. 2B and 2C, in particular, FIG. 2B illustrates the applicator 100 in an unactuated state. Axial movement of the stick/plunger 126 is limited by the radially outwardly extending flange 136 of the stick/plunger, and the surface 134 of the barrel 102. That is, the possible movement of the stick/plunger 126 within the barrel 102 is X as illustrated in FIG. 2B. Referring again to FIG. 2B, the distance that the stick/plunger 126 may travel axially before engaging the actuator assembly 156 is Y. The actuator assembly 156 likewise being axially movable, axial movement of the actuator assembly 156 is limited by the distance Z between the distal end 168 of the actuator 158 and an internal distal surface 166 of the tip 104 (see FIGS. 2E-2F). That is, in the illustrated embodiment, distance X is at least as great as distance Y plus distance Z.

In use, the operator depressed the engagement surface 128 of the drive stick/plunger 126 to axially advance the drive stick/plunger 126 within the barrel 102, the drive stick/plunger 126 traveling distance Y before engaging the actuator assembly 156. As the operator continues to depress the engagement surface 128 of the drive stick/plunger 126, the drive stick 126 likewise axially advances the actuator assembly 156 relative to the tip 104 and the needle/conduit 106 until the distal end 168 of the actuator 158 engages the internal distal surface 166 of the tip 104, that is, distance Z. In this way, the probe 160 is advanced through the needle/conduit 106 to dispense any medicament contained within the needle/conduit 106.

It will be appreciated that the foregoing description provides examples of the disclosed system and technique. However, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated.

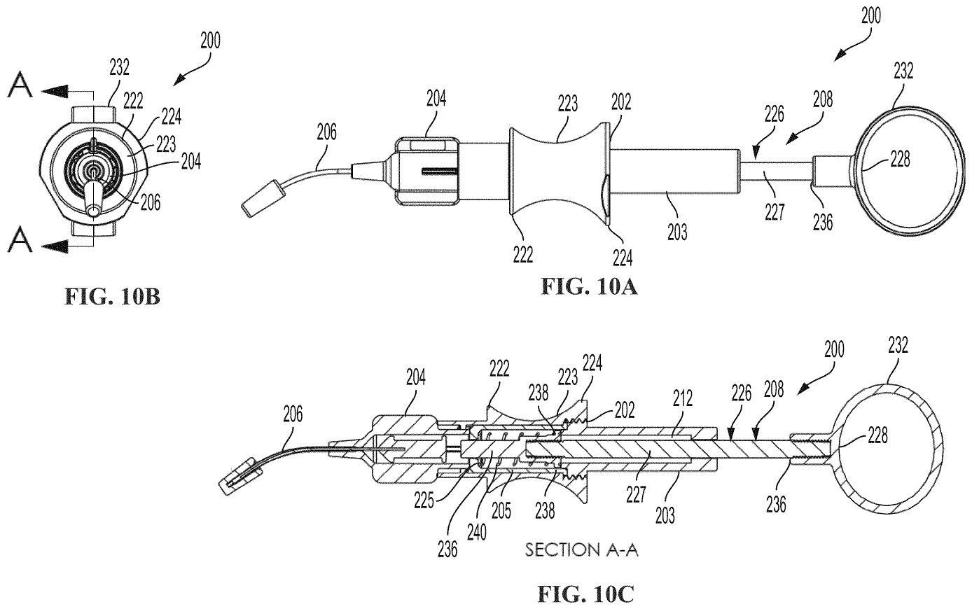

It will be appreciated that the applicator 100 for administration of a medicament such as a hydrogel or powder to a surface may differ in construction and dimension. For example, in order to facilitate fabrication, molding or assembly of the device, various components of the applicator 100 may be of alternative construction. An exemplary alternative embodiment of the applicator 200 is illustrated in FIGS. 10A-10C. The applicator 200 includes a barrel 202, to which a tip 204 having a delivery needle/conduit 206 is coupled.

A shield 254 may be provided to cover the end on the needle/conduit 206 in order to prevent an inadvertent contact or contamination with the tip of the needle/conduit 206, and to inhibit inadvertent dispensing of the medicament contained within the needle/conduit 206. An actuation assembly 208 is provided to facilitate delivery of the medicament through the delivery needle/conduit 206.

In the embodiment of FIGS. 10A-10C, the barrel 202 is a multipart structure that includes a first barrel section 203, and a second barrel section 223 that includes generally radially extending flanges 222, 224. The tip 204 may be coupled to the second barrel section 223 by any appropriate arrangement. Likewise, the first and second barrel sections 203, 223 may be coupled together by any arrangement. By way of example only, they may be coupled together by a splined or threaded arrangement. The barrel 202 may further be provided with a generally cylindrical structure 225 received within an internal chamber of the second barrel section 223. In this way, as the first barrel section 203 is coupled to the second barrel section 223, the generally cylindrical structure 225 is held in place within the internal chamber of the second barrel section 223.

A drive stick/plunger 226 likewise may be formed as a multipart structure. The drive stick/plunger 226 may include an elongated rod 227, with coupled engagement element 232 presenting an engagement surface 228, as well as an elongated drive element 230. The elongated rod 227, engagement element 232, and elongated drive element 230 may be coupled together by any appropriate arrangement, such as threaded or splined arrangements. It will be appreciated that, in assembly, the elongated drive element 230 may be assembled to the elongated rod 227 and positioned within the first barrel section 203. Further, a biasing element 240, such as a spring, may be positioned within the generally cylindrical structure 225 within the second barrel section 223 prior to coupling the first barrel section 203 to the second barrel section 223. In this way, the elongated drive element 230 may present a radially extending surfaces or flanges 238 that center or align the drive stick/plunger 226 along the drive axis within the barrel 202. Further the biasing element 240 is retained between a radially extending surface of the generally cylindrical structure 225 of the barrel 202 and the radially extending surfaces or flanges 238 of the drive stick/plunger 226. Further, it will be appreciated that radially extending surfaces or flanges 238 and radially extending surfaces 236 of the coupled engagement element 232 may be configured to engage the surfaces of the barrel 202, for example, the first barrel section 203, in order to limit axial travel of the drive stick 226 within the longitudinally extending internal chamber 212 of the barrel 202.

Other aspects of the device 200 may likewise be of varied designs so long as the techniques may be carried out with the device. It will be appreciated that the foregoing description provides examples of the disclosed system and technique. However, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated.

### Aspects of the Disclosure

Aspects, including embodiments, of the invention described herein may be beneficial alone or in combination, with one or more other aspects or embodiments. Without limiting the foregoing description, certain non-limiting aspects of the disclosure are enumerated below. As will be apparent to one of ordinary skill in the art upon reading this disclosure, each enumerated aspect may be used or combined with any of the preceding or following enumerated aspects. This is intended to provide support for all such combinations of aspects without limitation to combinations of aspects explicitly provided below:

(1) In aspect (1) is presented a composition comprising chitosan particles having a particle size distribution with a mode of from about 0.1 μm to about 100 μm.

(2) In aspect (2) is presented the composition of aspect 1, wherein the composition comprises up to 30 wt. % water.

(3) In aspect (3) is presented the composition of aspect 1 or 2, wherein the composition comprises less than 5 wt. % water.

(4) In aspect (4) is presented the composition of any one of aspects 1-3, wherein the composition comprises less than 1 wt. % water.

(5) In aspect (5) is presented the composition of any one of aspects 1-4, wherein the chitosan has a degree of deacetylation from about 50% to about 100% relative to chitin.

(6) In aspect (6) is presented the composition of any one of aspects 1-5, wherein the chitosan has a degree of deacetylation from about 80% to about 100% relative to chitin.

(7) In aspect (7) is presented the composition of any one of aspects 1-6, wherein the chitosan has a degree of deacetylation from about 80% to about 99% relative to chitin.

(8) In aspect (8) is presented the composition of any one of aspects 1-7, wherein the chitosan has a degree of deacetylation from about 85% to about 99% relative to chitin.

(9) In aspect (9) is presented the composition of any one of aspects 1-8, wherein the chitosan has a weight average molecular weight from about 1 kDa to about 500 kDa.

(10) In aspect (10) is presented the composition of any one of aspects 1-9, wherein the chitosan has a weight average molecular weight from about 1 kDa to about 50 kDa.

(11) In aspect (11) is presented the composition of any one of aspects 1-10, wherein the chitosan has a weight average molecular weight from about 1 kDa to about 25 kDa.

(12) In aspect (12) is presented the composition of any one of aspects 1-11, wherein the chitosan has a weight average molecular weight from about 4 kDa to about 20 kDa.

(13) In aspect (13) is presented the composition of any one of aspects 1-12, wherein the chitosan particles have a particle size distribution with a mode of from about 1 μm to about 30 μm.

(14) In aspect (14) is presented the composition of any one of aspects 1-13, wherein the chitosan particles have a particle size distribution with a mode of from about 1 μm to about 10 μm.

(15) In aspect (15) is presented the composition of any one of aspects 1-14, wherein the chitosan particles have a particle size distribution with a mode of from about 3 μm to about 5 μm.

(16) In aspect (16) is presented the composition of any one of aspects 1-15, wherein the chitosan particles have a d10 of at most about 5 μm.

(17) In aspect (17) is presented the composition of any one of aspects 1-16, wherein the chitosan particles have a d10 of at most about 4 μm.

(18) In aspect (18) is presented the composition of any one of aspects 1-17, wherein the chitosan particles have a d10 of at most about 3 μm.

(19) In aspect (19) is presented the composition of any one of aspects 1-18, wherein the chitosan particles have a d50 of at most about 10 μm.

(20) In aspect (20) is presented the composition of any one of aspects 1-19, wherein the chitosan particles have a d50 of at most about 8 μm.

(21) In aspect (21) is presented the composition of any one of aspects 1-20, wherein the chitosan particles have a d50 of at most about 5 μm.

(22) In aspect (22) is presented the composition of any one of aspects 1-21, wherein the chitosan particles have a d90 of at most about 20 μm.

(23) In aspect (23) is presented the composition of any one of aspects 1-22, wherein the chitosan particles have a d90 of at most about 15 μm.

(24) In aspect (24) is presented the composition of any one of aspects 1-23, wherein the chitosan particles have a d90 of at most about 10 μm.

(25) In aspect (25) is presented the composition of any one of aspects 1-24, wherein the chitosan particles are obtained by spray drying, ball milling, jet milling, cryo-milling, lyophilization, or microfluidization.

(26) In aspect (26) is presented the composition of any one of aspects 1-25, wherein the chitosan particles are obtained by spray drying.

(27) In aspect (27) is presented the composition of any one of aspects 1-26, wherein the chitosan (a) is esterified, sulfated, sulfonated, carboxymethylated, alkylated, sulfonylated, alkanoylated, crosslinked, or any combination thereof or (b) is in the form of a pharmaceutically acceptable acid addition salt.

(28) In aspect (28) is presented the composition of any one of aspects 1-27, wherein the chitosan is in the form of an acid addition salt which is hydrochloride, hydrobromide, hyaluronate, sulfate, phosphate, nitrate, formate, acetate, lactate, succinate, citrate, tartrate, or ascorbate.

(29) In aspect (29) is presented the composition of any one of aspects 1-28, wherein the chitosan is in the form of a hydrochloride salt.

(30) In aspect (30) is presented the composition of any one of aspects 1-29, wherein the composition is in the form of a powder, a pellet, a paste, or a bioadhesive gel.

(31) In aspect (31) is presented the composition of any one of aspects 1-30,

wherein the composition further comprises a therapeutically effective amount of an additional agent which is a local anesthetic, an antiseptic, an analgesic, an anti-inflammatory agent, an antibacterial agent, an anti-infective agent, or a combination thereof.

(32) In aspect (32) is presented the composition of aspect 31, wherein the composition comprises an antibacterial agent.

(33) In aspect (33) is presented the composition of aspect 32, wherein the antibacterial agent comprises a tetracycline.

(34) In aspect (34) is presented the composition of aspect 32, wherein the antibacterial agent comprises minocycline or a pharmaceutically acceptable salt thereof.

(35) In aspect (35) is presented the composition of aspect 32, wherein the antibacterial agent comprises minocycline hydrochloride.

(36) In aspect (36) is presented the composition of aspect 32, wherein the antibacterial agent is effective in treating an infection by one or more of Porphyromonas gingivalis, Prevotella intermedia, Fusobacterium nucleatum, Eikenella corrodens, and Actinobacillus actinomycetemcomitans.

(37) In aspect (37) is presented the composition of any one of aspects 31-36, wherein the chitosan particles release the additional agent into the periodontal pocket of the patient.

(38) In aspect (38) is presented the composition of any one of aspects 1-37, wherein the composition comprises a pharmaceutically acceptable carrier.

(39) In aspect (39) is presented the composition of aspect 38, wherein the carrier comprises a binder, a glidant, a dispersant, a disintegrant, an excipient, a lubricant, or a combination thereof.

(40) In aspect (40) is presented the composition of aspect 38 or 39, wherein the carrier comprises a buffer, a preservative, a flavor enhancer, a coloring agent, or a combination thereof.

(41) In aspect (41) is presented the composition of aspect 39, wherein the binder comprises microcrystalline cellulose, gum tragacanth, gelatin, or a combination thereof; the dispersant comprises a surfactant; the disintegrant comprises alginic acid, Primogel, corn starch, or a combination thereof; the excipient comprises a filler, starch, lactose, or a combination thereof; the lubricant comprises magnesium stearate; and the glidant comprises colloidal silicon dioxide.

(42) In aspect (42) is presented the composition of any one of aspects 1-30, wherein the composition consists essentially of the chitosan particles.

(43) In aspect (43) is presented the composition of any one of aspects 1-30, wherein the composition consists essentially of the chitosan particles and water.

(44) In aspect (44) is presented a method for treating an oral condition in a patient, the method comprising administering to the patient the composition of any one of aspects 1-43 to treat the oral condition.

(45) In aspect (45) is presented a method for treating an oral condition in a patient, the method comprising administering to the patient a therapeutically effective amount of a composition comprising or consisting essentially of the chitosan particles, to treat the oral condition.

(46) In aspect (46) is presented the method of aspect 44 or 45, wherein the method comprises subgingivally administering to the patient a therapeutically effective amount of the composition.

(47) In aspect (47) is presented the method of aspect 46, wherein the subgingival administration comprises inserting the composition into a periodontal pocket of the patient.

(48) In aspect (48) is presented the method of aspect 47, wherein the composition is retained in a periodontal pocket of the patient for at least or up to about 24 hours.

(49) In aspect (49) is presented the method of aspect 47, wherein the composition is retained in a periodontal pocket of the patient for at least or up to about 3 days.

(50) In aspect (50) is presented the method of aspect 47, wherein the composition is retained in a periodontal pocket of the patient for at least or up to about 14 days.

(51) In aspect (51) is presented the method of aspect 44 or 45, wherein the method comprises topically administering the composition to a tissue in an oral cavity of the patient.

(52) In aspect (52) is presented the method of aspect 51, wherein the composition is administered as a wound dressing.

(53) In aspect (53) is presented the method of aspect 44 or 45, wherein the method comprises sub-dermally administering the composition within an oral wound of the patient.

(54) In aspect (54) is presented the method of aspect 53, wherein the composition is sutured within the oral wound of the patient.

(55) In aspect (55) is presented the method of any one of aspects 44-54, wherein the oral condition is associated with periodontitis, peri-implantitis, peri-implant mucositis, or a combination thereof.

(56) In aspect (56) is presented the method of any one of aspects 44-55, wherein the oral condition comprises infection, inflammation, bleeding, suppuration, bone loss, biofilm, gum recession, tooth decay, pocket depth increase, or a combination thereof.

(57) In aspect (57) is presented the method of any one of aspects 44-56, wherein the composition is administered as an adjunct to a scaling or a root planing procedure, a periodontal flap surgery, a gingival graft, implant placement, a bone graft, or an extraction procedure.

(58) In aspect (58) is presented the method of any one of aspects 45-57, wherein the chitosan particles have a particle size distribution with a mode of from about 1 μm to about 100 μm.

(59) In aspect (59) is presented the method of any one of aspects 45-58, wherein the composition comprises up to 30 wt. % water.

(60) In aspect (60) is presented the method of any one of aspects 45-59, wherein the composition comprises less than 5 wt. % water.

(61) In aspect (61) is presented the method of any one of aspects 45-60, wherein the composition comprises less than 1 wt. % water.

(62) In aspect (62) is presented the method of any one of aspects 45-61, wherein the chitosan has a degree of deacetylation from about 50% to about 100% relative to chitin.

(63) In aspect (63) is presented the method of any one of aspects 45-62, wherein the chitosan has a degree of deacetylation from about 80% to about 100% relative to chitin.

(64) In aspect (64) is presented the method of any one of aspects 45-63, wherein the chitosan has a degree of deacetylation from about 80% to about 99% relative to chitin.

(65) In aspect (65) is presented the method of any one of aspects 45-64, wherein the chitosan has a degree of deacetylation from about 85% to about 99% relative to chitin.

(66) In aspect (66) is presented the method of any one of aspects 45-65, wherein the chitosan has a weight average molecular weight from about 1 kDa to about 500 kDa.

(67) In aspect (67) is presented the method of any one of aspects 45-66, wherein the chitosan has a weight average molecular weight from about 1 kDa to about 50 kDa.

(68) In aspect (68) is presented the method of any one of aspects 45-67, wherein the chitosan has a weight average molecular weight from about 1 kDa to about 25 kDa.

(69) In aspect (69) is presented the method of any one of aspects 45-68, wherein the chitosan has a weight average molecular weight from about 4 kDa to about 20 kDa.

(70) In aspect (70) is presented the method of any one of aspects 45-69, wherein the chitosan particles have a particle size distribution with a mode of from about 1 μm to about 30 μm.

(71) In aspect (71) is presented the method of any one of aspects 45-70, wherein the chitosan particles have a particle size distribution with a mode of from about 1 μm to about 10 μm.

(72) In aspect (72) is presented the method of any one of aspects 45-71, wherein the chitosan particles have a particle size distribution with a mode of from about 3 μm to about 5 μm.

(73) In aspect (73) is presented the method of any one of aspects 45-72, wherein the chitosan particles have a d10 of at most about 5 μm.

(74) In aspect (74) is presented the method of any one of aspects 45-73, wherein the chitosan particles have a d10 of at most about 4 μm.

(75) In aspect (75) is presented the method of any one of aspects 45-74, wherein the chitosan particles have a d10 of at most about 3 μm.

(76) In aspect (76) is presented the method of any one of aspects 45-75, wherein the chitosan particles have a d50 of at most about 10 μm.

(77) In aspect (77) is presented the method of any one of aspects 45-76, wherein the chitosan particles have a d50 of at most about 8 μm.

(78) In aspect (78) is presented the method of any one of aspects 45-77, wherein the chitosan particles have a d50 of at most about 5 μm.

(79) In aspect (79) is presented the method of any one of aspects 45-78, wherein the chitosan particles have a d90 of at most about 20 μm.

(80) In aspect (80) is presented the method of any one of aspects 45-79, wherein the chitosan particles have a d90 of at most about 15 μm.

(81) In aspect (81) is presented the method of any one of aspects 45-80, wherein the chitosan particles have a d90 of at most about 10 μm.

(82) In aspect (82) is presented the method of any one of aspects 45-81, wherein the chitosan particles are obtained by spray drying, ball milling, jet milling, cryo-milling, lyophilization, or microfluidization.

(83) In aspect (83) is presented the method of any one of aspects 45-82, wherein the chitosan particles are obtained by spray drying.

(84) In aspect (84) is presented the method of any one of aspects 45-83, wherein the chitosan (a) is esterified, sulfated, sulfonated, carboxymethylated, alkylated, sulfonylated, alkanoylated, crosslinked, or any combination thereof or (b) is in the form of a pharmaceutically acceptable acid addition salt.

(85) In aspect (85) is presented the method of any one of aspects 45-84, wherein the chitosan is in the form of an acid addition salt which is hydrochloride, hydrobromide, hyaluronate, sulfate, phosphate, nitrate, formate, acetate, lactate, succinate, citrate, tartrate, or ascorbate.

(86) In aspect (86) is presented the method of any one of aspects 45-85, wherein the chitosan is in the form of a hydrochloride salt.

(87) In aspect (87) is presented the method of any one of aspects 45-86, wherein the composition is in the form of a powder, a pellet, a paste, or a bioadhesive gel.

(88) In aspect (88) is presented the method of any one of aspects 45-87, wherein the composition further comprises a therapeutically effective amount of an additional agent which is a local anesthetic, an antiseptic, an analgesic, an anti-inflammatory agent, an antibacterial agent, an anti-infective agent, or a combination thereof.

(89) In aspect (89) is presented the method of aspect 88, wherein the composition comprises an antibacterial agent.

(90) In aspect (90) is presented the method of aspect 89, wherein the antibacterial agent comprises a tetracycline.

(91) In aspect (91) is presented the method of aspect 89, wherein the antibacterial agent comprises minocycline or a pharmaceutically acceptable salt thereof.

(92) In aspect (92) is presented the method of aspect 89, wherein the antibacterial agent comprises minocycline hydrochloride.

(93) In aspect (93) is presented the method of aspect 89, wherein the antibacterial agent is effective in treating an infection by one or more of Porphyromonas gingivalis, Prevotella intermedia, Fusobacterium nucleatum, Eikenella corrodens, and Actinobacillus actinomycetemcomitans.

(94) In aspect (94) is presented the method of any one of aspects 88-93, wherein the chitosan particles release the additional agent into a tissue in the oral cavity, such as the tissue of a periodontal pocket, of the patient.

(95) In aspect (95) is presented the method of any one of aspects 45-94, wherein the composition comprises a pharmaceutically acceptable carrier.

(96) In aspect (96) is presented the method of aspect 95, wherein the carrier comprises a binder, a glidant, a dispersant, a disintegrant, an excipient, a lubricant, or a combination thereof.

(97) In aspect (97) is presented the method of aspect 95 or 96, wherein the carrier comprises a buffer, a preservative, a flavor enhancer, a coloring agent, or a combination thereof.

(98) In aspect (98) is presented the method of aspect 96, wherein the binder comprises microcrystalline cellulose, gum tragacanth, gelatin, or a combination thereof; the dispersant comprises a surfactant; the disintegrant comprises alginic acid, Primogel, corn starch, or a combination thereof; the excipient comprises a filler, starch, lactose, or a combination thereof, the lubricant comprises magnesium stearate; and the glidant comprises colloidal silicon dioxide.

(99) In aspect (99) is presented the method of any one of aspects 45-87, wherein the composition consists essentially of the chitosan particles.

(100) In aspect (100) is presented the method of any one of aspects 45-87, wherein the composition consists essentially of the chitosan particles and water.

(101) In aspect (101) is presented the method of any one of aspects 44-100, wherein the therapeutically effective amount of the composition comprises at least about 0.01 mg of the chitosan particles.

(102) In aspect (102) is presented the method of any one of aspects 44-101, wherein the therapeutically effective amount of the composition comprises from about 0.01 mg to about 1 g of the chitosan particles.

(103) In aspect (103) is presented the method of any one of aspects 44-102, wherein the therapeutically effective amount of the composition comprises from about 0.01 mg to about 500 mg of the chitosan particles.

(104) In aspect (104) is presented the method of any one of aspects 44-103, wherein the therapeutically effective amount of the composition comprises from about 0.01 mg to about 100 mg of the chitosan particles.

(105) In aspect (105) is presented the method of any one of aspects 44-104, comprising administering the composition by way of a unit dose cartridge comprising a stainless needle attached to a plastic or metal tip, wherein the tip is inserted into the periodontal pocket or wound and the composition is expelled through the tip to administer the composition.

(106) In aspect (106) is presented the method of aspect 105, wherein the unit dose cartridge is a disposable unit dose cartridge.

(107) In aspect (107) is presented the method of any one of aspects 44-104, comprising administering the composition by way of a multi dose cartridge comprising a stainless needle attached to a plastic or metal tip, wherein the tip is inserted into the periodontal pocket or wound and the composition is expelled through the tip to administer the composition.

(108) In aspect (108) is presented the method of aspect 107, wherein the multi dose cartridge is a disposable multi dose cartridge.

(109) In aspect (109) is presented the method of any one of aspects 44-104, comprising administering the composition by way of an applicator.

(110) In aspect (110) is presented the method of aspect 109, wherein the applicator comprises any combination of at least a portion of the features described in FIGS. 1-8.

(111) In aspect (111) is presented the method of any one of aspects 44-104, comprising compacting the composition into a pellet and inserting the pellet into an oral cavity of the patient.

(112) In aspect (112) is presented the method of any one of aspects 44-104, comprising spreading the composition as a paste in an oral cavity of the patient.

(113) In aspect (113) is presented an applicator comprising any combination of at least a portion of the features described in FIGS. 1-8.

### EXAMPLES

The following examples further illustrate the invention but should not be construed as in any way limiting its scope.

### Example 1

This example provides a method for preparing an exemplary composition of the invention.

Chitosan lactate having approximately 95% deacetylation was added to an aqueous mixture containing glacial acetic acid to provide an aqueous chitosan mixture containing approximately 1% chitosan and approximately 1% acetic acid. The resulting mixture was blended with an immersion blender for 5 to 10 minutes until a foamy solution was formed. The foamy solution was stored overnight and the resulting solution was spray dried with the following parameters.
Fan: 100.0
Heating: 160.0° C.
Peristaltic Pump: 25
Needle: 6 s

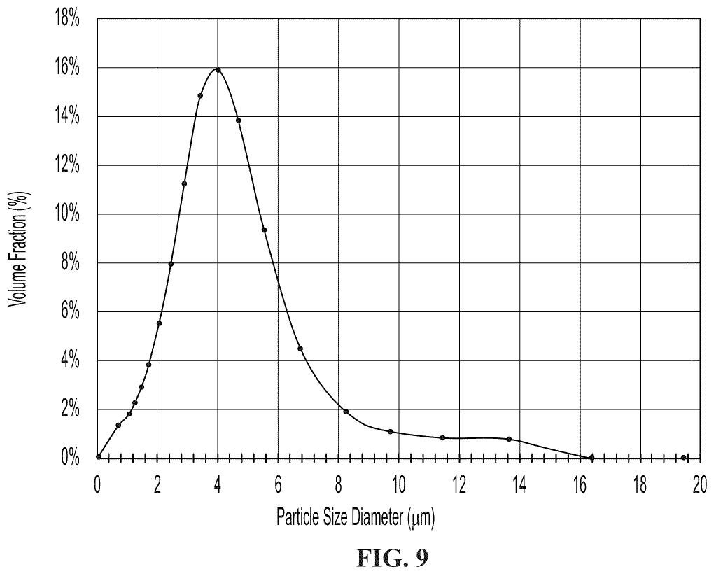

The spray dried chitosan was collected and stored as a dry powder. The particle size distribution of the powder was analyzed and the results are set forth in FIG. 9. As is apparent from the results set forth in FIG. 9, the powder comprising chitosan particles had a particle size distribution with a mode of about 4 μm.

### Example 2

This example provides an evaluation of the antimicrobial susceptibility of select organisms by measuring the Minimal Inhibitory Concentration (MIC) and Minimal Bactericidal Concentration (MBC) of a composition of Example 1.

The MIC endpoint is the lowest concentration of an antimicrobial agent that prevents visible growth of a microorganism. The MBC is the minimal concentration of an antimicrobial agent needed to kill most (i.e., ≥99.9%) of the viable microorganisms.

For this study, the composition of Example 1 was used to treat Aggregatibacter actinomycetemcomitans (ATCC 33384), Escherichia coli (ATCC 11229), Pseudomonas aeruginosa (ATCC 15442), Porphyromonas gingivalis (ATCC 33277), and Staphylococcus aureus ATCC 6538.

All isolates were obtained and frozen stock stored at −70° C. Seed-lot culture maintenance techniques were used so that the viable microorganisms used for inoculation were not more than five passages removed from the original master seed lot. E. coli, S. aureus, and P. aeruginosa were cultured onto Tryptic Soy Agar with 5% Sheep Blood (SBA) and incubated for 18-24 hours at 35±1° C. Aggregatibacter actinomycetemcomitans was propagated on Tryptic Soy Agar with 5% Sheep Blood (SBA) for 24±2 hours at 37±1° C. in a 4% to 7% CO₂ atmosphere. Porphyromonas gingivalis was propagated on pre-reduced and pre-warmed Brucella Blood Agar for 48±2 hours at 37±1° C. in an anaerobic atmospheric condition.

The inoculum was prepared by making a suspension of isolated colonies from the agar plate in 0.85% saline. Once the test suspension was formed, the organism concentration was adjusted in the selected broth so that after inoculation using the Boekel Replicator (1 μL transfer/pin) each well contained approximately 5×10⁴ CFU (target: 2 to 8×10⁴ CFU). All adjusted suspensions were used for final inoculation within 15 minutes of preparation. E. coli, S. aureus, and P. aeruginosa test organism inoculum suspension was prepared in Cation-adjusted Mueller-Hinton II Broth (CAMHB) following the direct colony suspension method as outlined in the CLSI M07-A11 standard. The Aggregatibacter actinomycetemcomitans inoculum suspension was prepared in Haemophilus Test Medium Broth (HTM Broth) following the direct colony suspension method as outlined in the CLSI M11-A9 standard. The Porphyromonas gingivalis inoculum suspension was prepared in pre-reduced and pre-warmed Brucella broth following procedures outlined in the CLSI M11-A9 standard.

Populations of organisms were determined by preparing ten-fold serial dilutions of each challenge organism suspension and plating in duplicate using standard microbiological procedures. Aerobic isolates were plated onto Tryptic Soy Agar with 5% Sheep Blood (SBA) incubated at 35±1° C. for 18-24 hours. Aggregatibacter actinomycetemcomitans was plated onto Tryptic Soy Agar with 5% Sheep Blood (SBA) for 48±2 hours at 37±1° C. in a 4% to 7% CO₂ atmosphere. Porphyromonas gingivalis was plated on pre-reduced and pre-warmed Brucella Blood Agar for 48±2 hours at 37±1° C. in an anaerobic atmospheric condition. Colonies were enumerated and recorded as CFU/plate to determine viable organisms. Duplicate plates were averaged and multiplied by the dilution factor to calculate the challenge organism microbial population (CFU/well)

To assess the MIC, selected concentrations of the test product were prepared in the selected broth for each organism and 100 μL dispensed in triplicate into the wells of the microwell plates. Orientation of well locations and associated product concentrations were recorded on the appropriate data sheets. Sterility and growth control wells were included for each plate. Following preparation of the plates, individual wells (excluding sterility assessment wells) were inoculated using the Boekel Replicator with the challenge microorganism being evaluated. To prevent drying, each tray was fitted with a plastic cover and placed in a sealed plastic container before incubating. The inoculated microwell plates were incubated under the appropriate conditions. Aerobic organisms (i.e., E. coli, S. aureus, and P. aeruginosa): 35±1° C. for 16-20 hours in ambient air. Aggregatibacter actinomycetemcomitans: 37±1 for 20-24 hours in a 4% to 7% CO₂ atmosphere. Porphyromonas gingivalis: 37±° C. for 46-48 hours in an anaerobic atmospheric condition. Following incubation, the wells were observed for evidence of visible growth and the MIC endpoint determined for the microorganism. The results are summarized in Table 1.

TABLE 1
Summary of MIC Endpoints

| | A. Actinomy- | E. | P. | P. | S. |
|---|---|---|---|---|---|
| | cetemcomitans | Coli | aeruginosa | gingivalis | Aureus |
| | (ppm) | (ppm) | (ppm) | (ppm) | (ppm) |
| Chitosan | 78 | 625 | 2500 | 78 | 156 |
| Powder | | | | | |

To assess MBC, using a 10 μL loop, each MIC well was streaked for isolation to an appropriate agar and incubated under the appropriate conditions. Aerobic isolates (i.e., E. coli, S. aureus, and P. aeruginosa) were struck onto Tryptic Soy Agar with 5% Sheep Blood (SBA) incubated at 35±1° C. for 18-24 hours. Aggregatibacter actinomycetemcomitans was struck on Tryptic Soy Agar with 5% Sheep Blood (SBA) for 24±2 hours at 37±1° C. in a 4% to 7% CO₂ atmosphere. Porphyromonas gingivalis was struck on pre-reduced and pre-warmed Brucella Blood Agar for 48±2 hours at 37±1° C. in an anaerobic atmospheric condition. Following incubation, absence of visible growth on the agar surface of a plate from the lowest concentration determined the MBC for the microorganism. The results are summarized in Table 2.

TABLE 2
Summary of MBC Endpoints

| | A. Actinomy- | E. | P. | P. | S. |
|---|---|---|---|---|---|
| | cetemcomitans | Coli | aeruginosa | gingivalis | Aureus |
| | (ppm) | (ppm) | (ppm) | (ppm) | (ppm) |
| Chitosan | 78 | 625 | 10000 | 78 | 156 |
| Powder | | | | | |

As is apparent from the results set forth in Table 1, a composition of Example 1 prevents visible growth of Aggregatibacter actinomycetemcomitans (ATCC 33384), Escherichia coli (ATCC 11229), Pseudomonas aeruginosa (ATCC 15442), Porphyromonas gingivalis (ATCC 33277), and Staphylococcus aureus ATCC 6538. As is apparent from the results set forth in Table 2, a composition of Example 1 can be applied in reasonable amounts to kill most (i.e., ≥99.9%) of the Aggregatibacter actinomycetemcomitans (ATCC 33384), Escherichia coli (ATCC 11229), Pseudomonas aeruginosa (ATCC 15442), Porphyromonas gingivalis (ATCC 33277), and Staphylococcus aureus ATCC 6538. For example, only 78 ppm of a composition of Example 1 is needed to prevent visible growth and kill most of the Aggregatibacter actinomycetemcomitans (ATCC 33384), Escherichia coli (ATCC 11229), Pseudomonas aeruginosa (ATCC 15442), Porphyromonas gingivalis (ATCC 33277), and Staphylococcus aureus ATCC 6538.

Without wishing to be bound by any particular theory, it is believed that applying the chitosan particles directly, e.g., as a powder, pellet, or paste, will provide an amount of the active sufficient to prevent visible growth of antimicrobials, while maintaining the location of application as a result of the composition being applied.

### Example 3

This example provides a method for preparing an exemplary composition of the invention. Chitosan lactate having approximately 95% deacetylation was added to an aqueous mixture containing glacial acetic acid to provide an aqueous chitosan mixture containing approximately 2% (although the concentration of chitosan may be increased) chitosan and approximately 1% acetic acid. The resulting mixture was blended with an immersion blender for 5 to 10 minutes until a foamy solution was formed. The foamy solution was stored overnight and the resulting solution was spray dried with the following parameters.
Fan: 100.0
Heating: 160.0° C.
Peristaltic Pump: 25
Needle: 6 s

The spray dried chitosan was collected and stored as a dry powder.

### Example 4

This example provides a clinical evaluation of a composition of the invention in the form of a powder consisting essentially of (deacetylated) chitosan particles (microspheres).

Chitosan particles (1 mg) were administered subgingivally by depositing the particles into the periodontal pocket of patients using a disposable applicator (1 mg powder per cartridge tip) after undergoing a scaling and root planing procedure. Patients participating in the evaluation received a full mouth examination prior to the procedure to provide a baseline for probing depth, bleeding, gingival inflammation, and oral hygiene. Patients were instructed to avoid manipulating or touching the treatment areas after treatment and, for the first 10 days after treatment, to avoid interproximal cleaning devices and chewing sticky, hard, or crunchy foods such as gum, taffy, apples, raw carrots, etc. Patients were evaluated in follow-up visit approximately 30-days post treatment.

Inflammation was assessed as follows:
0: Normal gingiva-no inflammation and no discoloration or bleeding
1: Mild inflammation-slight change in color and slight edema, but no bleeding on probing
2: Moderate inflammation-redness, edema and glazing, bleeding on probing
3: Severe inflammation-marked redness and edema, ulceration with tendency to spontaneous bleeding

Oral hygiene was assessed as follows:
0: No observable plaque.
1: A thin layer of plaque; only detectable by scraping with a probe.
2: A moderate layer of plaque along the gingival margin and plaque is visible clinically.
3: Heavy plaque accumulation is detected at the gingival margin and in the interdental spaces.

The results for 36 patients are as follows.

The mean pocket depth before treatment was 5.64 mm. The mean pocket depth after treatment with the chitosan particles was 4.21 mm, resulting in an average reduction in pocket depth of 1.43 mm.

Bleeding on probing was observed for 98% of patients before treatment. Bleeding on probing decreased significantly to 31% of patients after treatment with the chitosan particles.

The mean inflammation before treatment was 2.16. The mean inflammation score decreased significantly to 0.96 after treatment with the chitosan particles. The mean oral hygiene score before treatment was 1.81. The mean oral hygiene score after decreased significantly to 0.89 after treatment with the chitosan particles.

Reduced edema with limited to no bleeding or suppuration was observed. Significant pocket depth reduction also was observed. Other clinical observations included a visible change in gum tissue, which was not edematous as in the initial visit, but rather firm and light pink in color. These results demonstrate that the composition of the invention significantly improves recovery following a scaling and root planing procedure.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The present invention also contemplates and provides compositions that “consist essentially of” and/or that “consist of” the combinations of components described herein, as well as methods that “consist essentially of” and/or that “consist of” the combinations of method steps described herein, as the transitional phrases “consists essentially of” and “consists of” are interpreted under U.S. patent law.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition consisting essentially of chitosan particles having a particle size distribution with a mode of from about 0.1 μm to about 100 μm.

2. The composition of claim 1, wherein the composition comprises no more than about 30 wt. % water.

3-5. (canceled)

6. The composition of claim 1, wherein the chitosan has a degree of deacetylation from about 80% to about 100% relative to chitin.

7-9. (canceled)

10. The composition of claim 1, wherein the chitosan has a weight average molecular weight from about 1 kDa to about 50 kDa.

11-12. (canceled)

13. The composition of claim 1, wherein the chitosan particles have a particle size distribution with a mode of from about 1 μm to about 30 μm.

14-15. (canceled)

16. The composition of claim 1, wherein the chitosan particles have a d10 of at most about 5 μm.

17-18. (canceled)

19. The composition of claim 1, wherein the chitosan particles have a d50 of at most about 10 μm.

20-21. (canceled)

22. The composition of claim 1, wherein the chitosan particles have a d90 of at most about 20 μm.

23-27. (canceled)

28. The composition of claim 1, wherein the chitosan is in the form of an acid addition salt which is hydrochloride, hydrobromide, hyaluronate, sulfate, phosphate, nitrate, formate, acetate, lactate, succinate, citrate, tartrate, or ascorbate.

29. The composition of claim 28, wherein the chitosan is in the form of a lactate salt.

30. The composition of claim 1, wherein the composition is in the form of a powder, a pellet, a paste, or a bioadhesive gel.

31-44. (canceled)

45. A method for treating an oral condition in a patient, the method comprising subgingivally administering to the patient a therapeutically effective amount of a composition comprising chitosan particles to treat the oral condition.

46. (canceled)

47. The method of claim 45, wherein the subgingival administration comprises inserting the composition into a periodontal pocket of the patient.

48. The method of claim 47, wherein the composition is retained in a periodontal pocket of the patient for at least about 24 hours.

49-54. (canceled)

55. The method of claim 45, wherein the oral condition is associated with periodontitis, peri-implantitis, peri-implant mucositis, or a combination thereof.

56. The method of claim 45, wherein the oral condition comprises infection, inflammation, bleeding, suppuration, gum recession, bone loss, biofilm, tooth decay, pocket depth increase, or a combination thereof.

57. The method of claim 45, wherein the composition is administered as an adjunct to a scaling or a root planing procedure, a periodontal flap surgery, a gingival graft, implant placement, a bone graft, or an extraction procedure.

58-60. (canceled)

61. The method of claim 45, wherein the therapeutically effective amount of the composition comprises from about 0.01 mg to about 100 mg of the chitosan particles.

62. The method of claim 45, comprising administering the composition by way of a unit dose or multi dose cartridge comprising a stainless needle attached to a plastic or metal tip, wherein the tip is inserted subgingivally and the composition is expelled through the tip to administer the composition.

63. The method of claim 62, wherein the unit dose or multi dose cartridge is disposable.

64-65. (canceled)

66. The method of claim 45, comprising administering the composition by way of an applicator.

67. The method of claim 66, wherein the applicator comprises any combination of at least a portion of the features described in FIGS. 1-8.

68. The method of claim 45, comprising compacting the composition into a pellet and inserting the pellet subgingivally to the patient.

69. (canceled)

70. An applicator comprising any combination of at least a portion of the features described in FIGS. 1-8.
